(19) United States
(12) Patent Application Publication
(10) Pub. No.: US 2026/0204401 A1
(43) Pub. Date: Jul. 16, 2026

(54) Methods and Systems for Artificial Intelligence-Based Data Standardization

(71) Applicant: SURVEY GUARD LLC, Mililani, HI (US)

(72) Inventor: Demetrius Kirk, Mililani, HI (US)

(21) Appl. No.: 19/447,198

(22) Filed: Jan. 13, 2026

Related U.S. Application Data
(60) Provisional application No. 63/744,383, filed on Jan. 13, 2025.

(51) Int. Cl. G16H 40/20

(52) U.S. Cl. CPC G16H 40/20; G16H 70/20

(57) ABSTRACT

Disclosed techniques, systems, and computer-readable media for artificial intelligence (AI) based data ingestion of different formats. The techniques ingest structured data via an application programming interface (API) in a standardized format and ingest unstructured data from various sources. The techniques process unstructured data to extract compliance metadata and normalize it into the standardized format. The techniques generate, by prompting an AI model with structured data and normalized compliance metadata, a determination of a compliance deficiency. The techniques associate the compliance deficiency with tags corresponding to one or more regulations. The techniques display, on a graphical user interface, interfaces including a pathway sub interface comprising structured data, normalized compliance metadata, and associated tags, wherein the pathway sub interface receives user selections or inputs to a digital pathway.

### CROSS-REFERENCE TO RELATED APPLICATIONS

This application claims priority to U.S. Application No. 63/744,383, titled SurveyGuard: AI-Powered Modular Compliance and Quality Improvement Platform for Post-Acute Care, filed Jan. 13, 2025, which is hereby incorporated by reference in its entirety.

### TECHNICAL FIELD

The present invention generally relates to artificial intelligence-driven data standardization. More particularly, the invention relates to identifying deficiencies, associating deficiencies with tags, and providing interactive dashboard interfaces for digital pathways.

### BACKGROUND

Medical facilities operate within a complex regulatory environment that requires continuous monitoring, documentation, and corrective action to maintain compliance with federal and state standards. These facilities generate substantial volumes of compliance-related data across multiple domains. The compliance-related data originates from diverse sources creating a fragmented information landscape that impedes automated data processing, cross-source correlation, and compliance analysis.

Approaches for managing regulatory compliance face several technical limitations. Paper-based processes, isolated spreadsheets, and disconnected software systems prevent facilities from aggregating compliance data resulting in missed deadlines, incomplete documentation, and inconsistent practices across locations. Additionally, existing approaches do not adapt to changing facility conditions. Multi-facility organizations face particular challenges in achieving consistent oversight across geographically dispersed locations.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, an artificial intelligence (AI) based system configured to ingest data of different formats is provided. The system comprises one or more processors. The system comprises an AI model programmatically accessible by the one or more processors. The system comprises an application programming interface (API) configured to receive structured data comprising a standardized format. The system comprises one or more memories communicatively coupled to the one or more processors storing computing instructions that, when executed by the one or more processors, cause the one or more processors to ingest, as received via the API, the structured data. The computing instructions cause the one or more processors to ingest unstructured data. The computing instructions cause the one or more processors to process the unstructured data to extract compliance metadata. The computing instructions cause the one or more processors to normalize the compliance metadata into the standardized format of the structured data. The computing instructions cause the one or more processors to generate, by prompting the AI model with the structured data and normalized compliance metadata, a determination of a compliance deficiency in (i) the structured data or (ii) the normalized compliance metadata. The computing instructions cause the one or more processors to associate the compliance deficiency with corresponding tags, wherein the tags correspond to one or more regulations. The computing instructions cause the one or more processors to display, on a graphical user interface (GUI), one or more interfaces including at least a pathway sub interface comprising the structured data, normalized compliance metadata, and associated tags, wherein the pathway sub interface is configured to receive user selections or inputs to a digital pathway.

According to other aspects of the present disclosure, the AI based system may include one or more of the following features. The computing instructions may further cause the one or more processors to determine based on (i) the user selections or inputs, (ii) structured data, and (iii) normalized compliance metadata, a risk score for at least one of a resident or a facility. The computing instructions may further cause the one or more processors to determine, based on the risk score and the user selections or inputs, a survey readiness metric. The computing instructions may further cause the one or more processors to generate, by prompting the AI model with the structured data and normalized compliance metadata, a plan of correction (POC). The computing instructions may further cause the one or more processors to generate, based on the risk score and the compliance deficiency, a risk alert, wherein the one or more interfaces includes a dashboard sub interface comprising the survey readiness metric, the POC, and the risk alert. The computing instructions may further cause the one or more processors to correlate diagnostic codes with historical citation patterns. The computing instructions may further cause the one or more processors to weight, based on the correlated diagnostic codes and historical citation patterns, the risk score. The computing instructions may further cause the one or more processors to prioritize, based on the weighted risk score, the digital pathway. The computing instructions may further cause the one or more processors to update, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database. The computing instructions may further cause the one or more processors to trigger, based on the associated tags, one or more compliance actions including at least one of (i) an audit, (ii) a risk score update, (iii) a quality improvement recommendation, or (iv) a mock survey. The computing instructions may further cause the one or more processors to generate an audit assessment tool based on the compliance deficiency or the user selections or inputs. Normalizing the compliance metadata into the standardized format may further comprise chunking the compliance metadata into segments, embedding, using an embedding model, the segments into vector representations, and aggregating, using retrieval-augmented generation (RAG), a prompt for the AI model including (i) relevant vector embeddings, (ii) a structured template, (iii) regulatory logic, and (iv) a metadata-specific prompt. The pathway sub interface may be configured to (i) enforce completion of a required field before the user selections or inputs and (ii) associate the user selections or inputs with the corresponding tags, and wherein the tags include at least one of an F-tag, a K-tag, or an E-tag. The POC may include systemic changes, monitoring plans, responsible roles, due dates, and evidence placeholders. The computing instructions may further cause the one or more processors to detect triggers within compliance issues. The computing instructions may further cause the one or more processors to escalate, based on a regulatory deadline, the compliance issues containing triggers. The standardized format may comprise at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

According to another aspect of the present disclosure, a method for artificial intelligence (AI) based data ingestion of different formats is provided. The method comprises ingesting, by one or more processors via an application programming interface (API), structured data comprising a standardized format. The method comprises ingesting, by the one or more processors, unstructured data. The method comprises processing, by the one or more processors, the unstructured data to extract compliance metadata. The method comprises normalizing, by the one or more processors, the compliance metadata into the standardized format of the structured data. The method comprises generating, by prompting an AI model with the structured data and normalized compliance metadata, a determination of a compliance deficiency in (i) the structured data or (ii) the normalized compliance metadata. The method comprises associating, by the one or more processors, the compliance deficiency with corresponding tags, wherein the tags correspond to one or more regulations. The method comprises displaying, on a graphical user interface (GUI), one or more interfaces including at least a pathway sub interface comprising the structured data, normalized compliance metadata, and associated tags, wherein the pathway sub interface is configured to receive user selections or inputs to a digital pathway.

According to other aspects of the present disclosure, the method may include one or more of the following features. The method may further comprise determining, based on (i) the user selections or inputs, (ii) structured data, and (iii) normalized compliance metadata, a risk score for at least one of a resident or a facility. The method may further comprise determining, based on the risk score and the user selections or inputs, a survey readiness metric. The method may further comprise generating, by prompting the AI model with the structured data and normalized compliance metadata, a plan of correction (POC). The method may further comprise generating, based on the risk score and the compliance deficiency, a risk alert, wherein the one or more interfaces includes a dashboard sub interface comprising the survey readiness metric, the POC, and the risk alert. The method may further comprise correlating diagnostic codes with historical citation patterns. The method may further comprise weighting, based on the correlated diagnostic codes and historical citation patterns, the risk score. The method may further comprise prioritizing, based on the weighted risk score, the digital pathway. The method may further comprise updating, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database. The method may further comprise triggering, based on the associated tags, one or more compliance actions including at least one of (i) an audit, (ii) a risk score update, (iii) a quality improvement recommendation, or (iv) a mock survey. The method may further comprise generating an audit assessment tool based on the compliance deficiency or the user selections or inputs. Normalizing the compliance metadata into the standardized format may further comprise chunking the compliance metadata into segments, embedding, using an embedding model, the segments into vector representations, and aggregating, using retrieval-augmented generation (RAG), a prompt for the AI model including (i) relevant vector embeddings, (ii) a structured template, (iii) regulatory logic, and (iv) a metadata-specific prompt. The pathway sub interface may be configured to (i) enforce completion of a required field before the user selections or inputs and (ii) associate the user selections or inputs with the corresponding tags, and wherein the tags include at least one of an F-tag, a K-tag, or an E-tag. The POC may include systemic changes, monitoring plans, responsible roles, due dates, and evidence placeholders. The method may further comprise detecting triggers within compliance issues. The method may further comprise escalating, based on a regulatory deadline, the compliance issues containing triggers. The standardized format may comprise at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

According to another aspect of the present disclosure, a tangible, non-transitory computer-readable medium storing instructions for artificial intelligence (AI) based data ingestion of different formats is provided. The instructions, when executed by one or more processors, cause the one or more processors to ingest, via an application programming interface (API), structured data comprising a standardized format. The instructions cause the one or more processors to ingest unstructured data. The instructions cause the one or more processors to process the unstructured data to extract compliance metadata. The instructions cause the one or more processors to normalize the compliance metadata into the standardized format of the structured data. The instructions cause the one or more processors to generate, by prompting an AI model with the structured data and normalized compliance metadata, a determination of a compliance deficiency in (i) the structured data or (ii) the normalized compliance metadata. The instructions cause the one or more processors to associate the compliance deficiency with corresponding tags, wherein the tags correspond to one or more regulations. The instructions cause the one or more processors to display, on a graphical user interface (GUI), one or more interfaces including at least a pathway sub interface comprising the structured data, normalized compliance metadata, and associated tags, wherein the pathway sub interface is configured to receive user selections or inputs to a digital pathway.

According to other aspects of the present disclosure, the tangible, non-transitory computer-readable medium may include one or more of the following features. The instructions may further cause the one or more processors to determine, based on (i) the user selections or inputs, (ii) structured data, and (iii) normalized compliance metadata, a risk score for at least one of a resident or a facility. The instructions may further cause the one or more processors to determine, based on the risk score and the user selections or inputs, a survey readiness metric. The instructions may further cause the one or more processors to generate, by prompting the AI model with the structured data and normalized compliance metadata, a plan of correction (POC). The instructions may further cause the one or more processors to generate, based on the risk score and the compliance deficiency, a risk alert, wherein the one or more interfaces includes a dashboard sub interface comprising the survey readiness metric, the POC, and the risk alert.

The foregoing general description of the illustrative embodiments and the following detailed description thereof are merely exemplary aspects of the teachings of this disclosure and are not restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates a block diagram of an artificial intelligence-based system for standardizing formats, according to aspects of the present disclosure.

FIG. 2A illustrates a block diagram of a flow for processing data of different formats, according to aspects of the present disclosure;

FIG. 2B illustrates a flowchart for an artificial intelligence processing flow depicting a data transformation pipeline, according to aspects of the present disclosure;

FIG. 3 illustrates a flowchart for a method of processing compliance data based on data format determination and deficiency identification, according to aspects of the present disclosure;

FIG. 4A illustrates a flowchart for a method of calculating risk scores and determining survey readiness with conditional compliance action triggering, according to aspects of the present disclosure;

FIG. 4B illustrates a flowchart for a method of processing unstructured data using retrieval-augmented generation and deficiency identification, according to aspects of the present disclosure;

FIG. 5A illustrates a sequence diagram representing a process for processing compliance-related data through a retrieval-augmented generation pipeline, according to aspects of the present disclosure.

FIG. 5B illustrates a sequence diagram representing a process for processing compliance-related data through a retrieval-augmented generation pipeline with optical character recognition and audit scheduling, according to aspects of the present disclosure;

FIG. 6 illustrates a flowchart for a method for processing entries and managing compliance actions based on trigger detection and deadline monitoring, according to aspects of the present disclosure;

FIG. 7 illustrates a flowchart for a method for processing structured and unstructured data to identify compliance deficiencies and display associated information on a graphical user interface, according to aspects of the present disclosure; and

FIG. 8 illustrates a block diagram of a multi-facility compliance and survey-readiness system depicting a hierarchical architecture for managing regulatory risk across multiple facilities, according to aspects of the present disclosure.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The present disclosure relates to artificial intelligence (AI) based data standardization techniques that may address data fragmentation challenges in healthcare settings. Compliance-relevant information in such settings may be spread across multiple disparate sources with no mechanism to connect the information into a unified format. For example, electronic medical records (EMR) systems, such as PointClickCare, MatrixCare, or other EMR platforms, may store clinical data in proprietary formats. Centers for Medicare & Medicaid Services (CMS) data sets may provide regulatory metrics in separate databases such as: Certification and Survey Provider Enhanced Reports (CASPER), which provide facilities with historical survey citations, quality measure trends, and comparative performance data used for compliance monitoring; 2567 statements which are official CMS Statement of Deficiencies forms issued by surveyors following a regulatory inspection that document specific violations and require formal corrective responses; and Five-Star ratings, which are a public quality rating system published by CMS that rates nursing homes on a scale of one to five stars based on health inspections, staffing, and quality measures. Staff logs including shift change notes and incident reports may capture operational observations. Grievance notebooks containing resident complaint forms and family concern documentation may record consumer feedback. Audit binders holding infection control audits and medication pass observations may document compliance assessments. Regional spreadsheets including multi-facility tracking documents and corporate compliance summaries may aggregate data across organizational hierarchies. These sources may operate in isolation with no mechanism to connect grievances to F-tags which are CMS federal regulatory citation categories that identify specific federal requirements for nursing home participation in Medicare and Medicaid programs; tie audits to Quality Assurance and Performance Improvement (QAPI) programs which are federally mandated continuous quality improvement programs that require facilities to systematically monitor care quality, identify problems, and implement corrective measures; or link clinical trends to regulatory risk.

The disclosed techniques may ingest data of different formats to address the fragmentation challenge. Structured data may include JavaScript Object Notation (JSON) data received from EMR application programming interfaces (APIs). Unstructured data may include Portable Document Format (PDF) files from scanned documents. Semi-structured data may include Comma Separated Value (CSV) exports from CMS databases. The system may process both structured and unstructured compliance data and use an AI model to identify compliance deficiencies in healthcare facilities (e.g., skilled nursing facilities, assisted living communities, long-term acute care hospitals, etc.). As described herein, a survey may refer to a regulatory inspection conducted by state or federal surveyors who visit healthcare facilities to assess compliance with applicable healthcare regulations, and survey readiness may refer to a facility's state of preparedness for such regulatory inspections.

The disclosed techniques may address the data fragmentation challenge through automated normalization, AI-driven analysis, and a unified compliance data model. Automated normalization may convert free-text observations into structured metadata fields that conform to a standardized format. AI-driven analysis may perform pattern recognition across clinical and regulatory data to identify relationships. The unified compliance data model may transform fragmented inputs into actionable compliance actions. Such actionable compliance actions may include prioritized risk alerts that highlight areas of concern, survey readiness dashboards that present compliance status metrics, and AI-generated plans of correction (POCs) that address identified deficiencies with structured remediation steps. As described herein, a POC refers to any corrective action or remediation workflow that the present techniques generate or assign to a user. In some examples, a POC may be a formal written remediation document that a healthcare facility must submit to regulatory agencies following a citation, detailing specific corrective actions the facility will take, systemic changes to prevent recurrence, responsible parties, monitoring procedures, and completion timelines. It will be understood that a POC is the corrective action framework and is not limited to only a post-survey or state-citation document. Similarly, it will be understood that while depicted in the various figures as associated with a specific module, POC and corrective actions may originate from any module based on the analysis of data, findings, trends, user activity, etc.. In some examples, POC may include any corrective action taken by the present techniques, and the various functionalities described herein are not limited to implementation by a single module or the specific architecture depicted, which is for illustrative purposes.

The disclosed techniques may provide technical advantages and improvements over other approaches to compliance data management through unified data processing, data retrieval mechanisms, automated regulatory tagging, and dynamic risk assessment.

The disclosed techniques may integrate structured and unstructured data processing within a unified computational pipeline. The disclosed techniques may ingest both data types, apply optical character recognition (OCR) to extract text from unstructured sources, and normalize the extracted compliance metadata into a standardized format compatible with the structured data. This unified processing may reduce the computational overhead associated with maintaining separate data processing pathways. For example, the unified processing may eliminate redundant memory allocation for parallel data transformation operations and may reduce processor cycles required for format conversion across multiple pipelines. The unified processing may also enable cross-source pattern detection that isolated systems cannot achieve. The normalization process may include chunking compliance metadata into segments, embedding the segments into vector representations using an embedding model, and aggregating a prompt using retrieval-augmented generation (RAG). The normalization process may transform incompatible data formats into a unified standardized format (e.g., JSON, eXtensible Markup Language (XML), CSV, Health Level Seven (HL7), etc.). The unified standardized format may reduce memory utilization by eliminating redundant data structures and may enable efficient AI model inference over a single normalized dataset rather than requiring separate processing pipelines for each data format.

The disclosed techniques may also apply RAG to transform compliance metadata into AI model inputs. The disclosed system may chunk extracted text into segments, convert the segments into vector embeddings (e.g., 1536-dimensional vector representations) using an embedding model, store the embeddings in a vector database, and retrieve relevant embeddings based on similarity search. The retrieved content may be combined with structured templates, regulatory logic, and domain-specific prompts before prompting an AI model (e.g., large language model (LLM)). This architecture may enable the AI model to generate context-aware outputs such as plans of correction (POCs) and compliance deficiency determinations without requiring model retraining for each new document type or regulatory requirement. The RAG approach may reduce inference latency compared to approaches that process entire document corpora by limiting the input token count to relevant retrieved segments rather than full documents. For example, the RAG approach may reduce processor load by retrieving relevant document chunks rather than processing thousands of pages, thereby decreasing memory consumption during model inference. The RAG approach may also improve output accuracy by grounding model responses in retrieved compliance content, which may reduce hallucination rates and improve the precision of generated compliance determinations. The disclosed techniques may further improve computational efficiency by correlating diagnostic codes with historical citation patterns to weight risk scores and prioritize digital pathways. The techniques may update the risk score, survey readiness metric, or vector database based on audit results, compliance deficiencies, or survey outcome data. This updating may enable the AI model to generate increasingly accurate compliance deficiency determinations without requiring retraining of the underlying model.

The disclosed techniques may further automate the association of identified compliance deficiencies with corresponding regulatory tags such as: F-tags; K-tags, which are CMS federal fire safety citation categories that identify specific Life Safety Code requirements for healthcare facility fire protection and safety systems; and E-tags, which are CMS emergency preparedness citation categories that identify specific requirements for facility emergency planning, policies, and procedures. Each tag corresponds to a specific regulatory requirement, and when a surveyor identifies a violation, the facility receives a citation referencing the applicable tag. The disclosed techniques may associate tags to programmatically link deficiencies based on the AI model output and the normalized compliance metadata. This automated tag association may enable downstream compliance actions to be triggered without manual intervention. For example, the automated tag association may automatically initiate audits, update risk scores, generate quality improvement recommendations, or schedule mock surveys (i.e., practice inspections conducted internally by facility staff to simulate actual regulatory surveys and identify potential deficiencies before official inspections occur) based on associated tags. The automated tag association may improve the process between deficiency identification and corrective action initiation thereby reducing processor idle time that would otherwise occur.

In conjunction with automated tag association, the disclosed techniques may calculate risk scores using diagnostic code weighting. The disclosed system may correlate diagnostic codes (e.g., International Classification of Diseases, 10th Revision (ICD-10) codes) with historical citation patterns to weight risk scores for residents and facilities. This weighting mechanism may prioritize digital pathways and compliance actions based on quantified risk levels rather than static thresholds. A risk scoring engine may update scores dynamically based on user inputs, audit results, compliance deficiencies, and survey outcome data. The dynamic updating may enable the system to adapt to changing facility conditions without requiring reconfiguration. The system may update the risk score, survey readiness metric, or vector database based on audit results, compliance deficiencies, or survey outcome data corresponding to previous compliance deficiency determinations. This updating may enable continuous improvement of compliance predictions without retraining of the underlying AI model.

The present techniques may enable a closed-loop workflow automation where mock survey findings lead to AI POC generation, then to assigned monitoring, smart audits, QAPI projects, and dashboard updates. The disclosed techniques may generate complete five-element POCs faster than other approaches (e.g., under 30 seconds). In some examples, CMS may require five elements for a compliant POC including identification of how the deficiency affected residents, a statement of the corrective action taken, identification of systemic changes to prevent recurrence, a monitoring plan to ensure the corrective action is effective, and target completion dates. Further, the disclosed techniques may also generate a complete audit tool faster than other approaches (e.g., in less than one minute). Digital pathways may complete in half the time of other approaches. The transitions between compliance stages may carry associated obligations, timers, owners, and evidence requirements that ensure compliance activities progress through defined stages with interpretability at each step. The unified architecture underlying these workflow transitions may reduce overall system memory footprint and processing requirements. The disclosed techniques may improve over prior approaches at least because the present techniques digitize CMS pathways, and integrate POCs, QAPI, grievances, dashboards, and risk scoring into a unified architecture that reduces overall system memory footprint and processing requirements. It should be understood that as described herein, a POC functions as a platform-wide corrective action framework that may be recommended, generated, updated, and tracked across the framework. In some examples, the depicted modules, described below, may serve as interfaces for creating and managing corrective actions, but the underlying corrective action construct may be accessible through the system as a whole, and corrective actions may originate from any module.

### Example AI-Based Data Standardization System

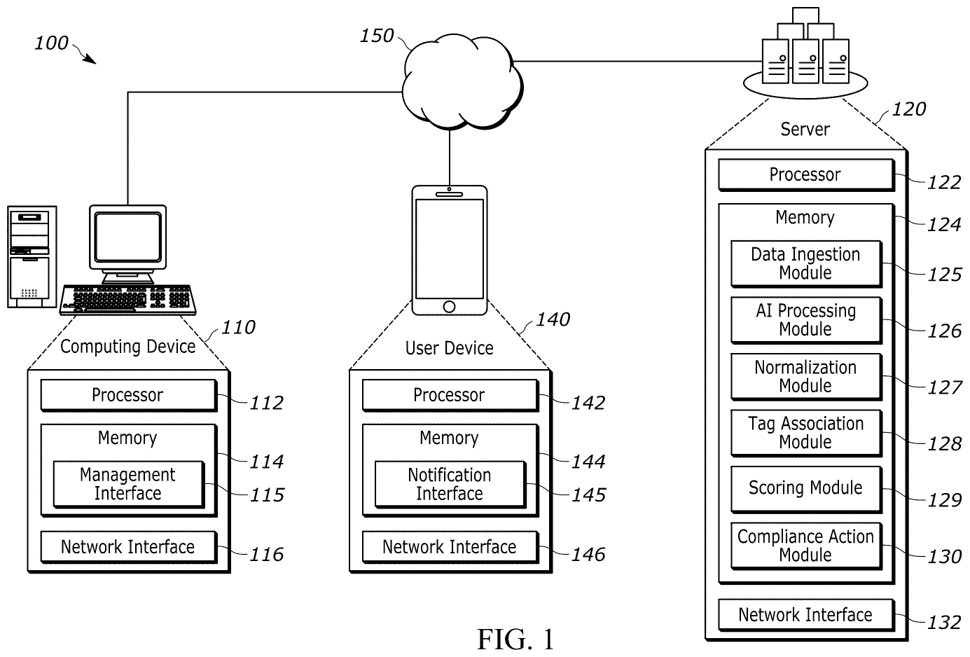

FIG. 1 depicts an artificial intelligence (AI) based system 100 configured to standardize data of different formats. The system 100 includes a computing device 110, a server 120, a user device 140, and a network 150. It should be understood that the system 100 is not limited by the specific components or architectures described herein but may include any suitable number, type, or configuration of components for implementing the techniques, examples, and/or embodiments of the present disclosure. As such, an additional or alternative device (e.g., the computing device 110 instead of the server 120, a cloud computing device in addition to the server 120, etc.) and/or component (e.g., data ingestion module 125, AI processing module 126, normalization module 127, tag association module 128, scoring module 129, compliance action module 130, etc.) may perform the functionality described herein (e.g., store a component in memory, execute software that performs one or more operations, etc.). In some examples, the computing device 110 and the server 120 may be part of the same computing device and/or system.

### Computing Device

The computing device 110 may be an individual computing device, a group (e.g., cluster) of multiple computing devices, or another suitable type of computing device or system (e.g., workstation, administration terminal, etc.). For example, the computing device 110 may be any suitable computing device (e.g., a laptop, desktop computer, workstation, etc.). In some examples, components of the computing device 110 may be embodied by virtual instances (e.g., using a cloud-based virtualization service). In such cases, the computing device 110 may be included in a remote data center (e.g., a cloud computing environment, a public cloud, a private cloud, or the like). In the example of FIG. 1, the computing device 110 includes a processor 112, a memory 114, a management interface 115, and a network interface 116.

The processor 112 may include any suitable number of processors and/or processor types. In some examples, the processor 112 includes one or more central processing units (CPUs), one or more graphics processing units (GPUs), one or more tensor processing units (TPUs), one or more field-programmable gate arrays (FPGAs), one or more application-specific integrated circuits (ASICs), and/or the like. Generally, the processor 112 comprises hardware configured to execute instructions (i.e., processor-executable code/instructions) stored in the memory 114.

The memory 114 may include any suitable memory type(s), including volatile memories (e.g., dynamic and/or static random-access memory (RAM)) and/or non-volatile memories (e.g., read-only memory (ROM), erasable programmable ROM (EPROM), electrically EPROM (EEPROM), NAND flash, and/or solid state drive(s) (SSD(s))), all or any of which are examples of non-transitory computer-readable media. In some examples, the memory 114 stores at least one of: an operating system; software components/modules (e.g., firmware, application(s), binary, source code, executable instructions, machine learning model(s)); transient data and/or code loaded and/or operated on by software component(s); and/or other suitable components/data.

The management interface 115 may include a display, such as a monitor, and a user input device, such as a keyboard, mouse, trackpad, gesture and/or biometric tracking device, or the like. The management interface 115 may enable administrative users to interact with a graphical user interface for configuration, monitoring, and control operations of the system 100. The network interface 116 comprises one or more hardware components that generally enable the computing device 110 to communicate via one or more networks (e.g., the network 150) with other components and/or devices of the system 100 and/or other suitable systems, devices, or combinations thereof. More specifically, the network interface 116 enables the computing device 110 to communicate with any component of the system 100 across the network 150. The network interface 116 may comprise hardware and/or software that operates according to at least one communication protocol of the network 150, described in further detail below.

### Server

The server 120 may be an individual server, a group of multiple servers, or another suitable type of device or group of devices. For example, the server 120 may be any suitable server device (e.g., rack server, tower server, blade server, mainframe, etc.). In some examples, the components of the server 120 may be embodied by virtual instances. In such cases, the server 120 may be included in a remote data center. In the example of FIG. 1, the server 120 includes a processor 122, a memory 124, a data ingestion module 125, an AI processing module 126, a normalization module 127, a tag association module 128, a scoring module 129, a compliance action module 130, and a network interface 132 substantially similar to that of the computing device 110, described above.

The memory 124 may include a set of computer-executable instructions (e.g., software) for compliance data processing operations (e.g., data ingestion module 125, AI processing module 126, normalization module 127, tag association module 128, scoring module 129, compliance action module 130). The memory 124 may be communicatively coupled to the processor 122 and may store computing instructions that, when executed by the processor 122, cause the processor 122 to perform the operations described herein. The memory 124 may additionally store other data, such as compliance databases, regulatory tag mappings, historical citation patterns, vector embeddings, and the like.

The data ingestion module 125 may receive and process compliance data from multiple sources to prepare the data for subsequent analysis. The data ingestion module 125 may be configured to receive structured data via an application programming interface (API) configured to receive structured data comprising a standardized format (e.g., JSON, XML, CSV, HL7). The data ingestion module 125 may also receive unstructured data from various sources including scanned documents, handwritten notes, and uploaded files. The data ingestion module 125 may segment input data by dividing the input into data segments for processing. This may include parsing structured data fields and extracting text from unstructured sources. The data ingestion module 125 may validate data to ensure integrity before passing the data to subsequent processing components. In some examples, the data ingestion module 125 may convert input data to a normalized encoding format to remove variations in character encoding across different data sources.

The AI processing module 126 may include an AI model programmatically accessible by the one or more processors (e.g., the processor 122). The AI processing module 126 may be configured to generate compliance deficiency determinations and POCs using a generative AI model (e.g., a language model). It should be understood that while depicted as a single module, the generation of POC's and corrective action functionality may be performed across a single module or a plurality of different modules, and corrective actions may originate from any module. In examples with a language model, the language model may be a transformer-based model trained to accept and analyze input text to generate output text. In some examples, the language model is a transformer-based machine-learned model (e.g., decoder-only or encoder-decoder architecture), such as an LLM, a multimodal LLM, and/or the like that operates upon and/or generates text along with one or more other types of content (e.g., images, video frames, and/or audio). The language model may comprise machine-learned model component(s), such as neural network(s), decision tree(s), and/or the like. The language model may receive a text prompt as input, process the text prompt, and output text content responsive to the text prompt. The language model may perform various natural language processing tasks as needed to understand a text query/prompt and generate a response to the text query/prompt.

In examples with a transformer-based model architecture, the language model may comprise an encoder that tokenizes the input and determines embeddings for the tokens, and a decoder that generates the output based at least in part on the embeddings. The language model may incorporate self-attention, cross-attention, and/or any suitable self-attention or attention mechanisms to facilitate more accurate output. In some examples, the language model may include different configurations of self-and/or cross-attention, followed by one or more neural networks (e.g., feedforward layer(s)), recurrent layer(s), aggregation layer(s) (e.g., using SoftMax, matrix multiplication, and/or other aggregation techniques), and/or the like. The language model may be a general-purpose model (e.g., trained on a wide array of publicly available datasets such as web pages, documents, etc., available via the Internet), such as generative pre-trained transformer (GPT) or a domain-specific model (e.g., trained and/or fine-tuned on custom and/or proprietary datasets), for example.

The language model may be trained by any suitable method (e.g., pre-training, fine-tuning, reinforcement learning from human feedback (RLHF), transfer learning, or zero/few/one-shot learning) using compliance datasets to generate high-quality compliance outputs. It should be understood that the language model may be locally stored in the server 120 using the memory 124, or may be cloud based (e.g., hosted by OpenAI®, Amazon Web Services® (AWS), or another suitable service or platform), and may be accessed by the computing device 110 and/or the server 120 via an API or the like. It should be appreciated that some examples may include combinations of the foregoing such as using a locally hosted language model in some scenarios and a different cloud-based generative model for other scenarios (i.e., for privacy reasons, computational efficiency, to optimize costs, etc.).

The AI processing module 126 may also support conversational and query-driven compliance. Users may submit questions regarding compliance risk or required actions through the notification interface 145, and the AI processing module 126 may determine responses based on aggregated data, regulatory logic, and historical outcomes. For example, a user may query what compliance risk exists following a particular event, what audits should be initiated, or what quality improvement activities are required. In some examples, a user may ask “What compliance risk exists following a fall in Room 201?” and the AI processing module 126 may analyze the resident's fall history, diagnoses, care plan, and facility citation patterns to generate a risk assessment. In some examples, a user may ask “What audits should be initiated after three infection control grievances?” and the AI processing module 126 may recommend specific infection control audits based on the grievance content and historical citation patterns. In some examples, a user may ask “What QAPI activities are required for our trends?” and the AI processing module 126 may generate QAPI project recommendations with prefilled goals, measures, and follow-up cadence. The AI processing module 126 may use normalized compliance and clinical data to determine risk, select appropriate compliance actions, and initiate those actions automatically, supporting decision-making rather than static reporting.

In some examples, the AI processing module 126 may operate as a platform-wide recommendation engine that continuously interprets findings and trends from all compliance modules, identifies triggers, and recommends actions. The recommendations may include generating or updating POCs, generating audit tools, assigning tasks and monitoring workflows, escalating issues based on regulatory deadlines, initiating QAPI projects, etc. This AI recommendation framework may operate across a plurality of modules to produce coordinated outputs. The AI recommendation engine may receive data from multiple compliance modules including mock surveys, audits, grievances, rounding, QAPI, and survey command center components, and may generate outputs including POC's as a system-wide framework and recommendations for audit tasks, monitoring activities, and QAPI projects that feed into real-time dashboards.

The normalization module 127 may normalize the extracted compliance metadata to prepare it for subsequent analysis. The normalization module 127 may be configured to convert compliance metadata extracted from unstructured data into standardized formats compatible with the structured data. The normalization module 127 may normalize data into formats including JSON, XML, CSV, or HL7. The normalization module 127 segments the extracted text by dividing the text into text segments (e.g., words, sentences, phrases, clauses, paragraphs, etc.). This may include tokenizing the text by segmenting the text into discrete units such as words or symbols, for example. An appropriate pre-processing technique may be employed by the normalization module.

The tag association module 128 may group identified compliance deficiencies with corresponding regulatory tags. The tag association module 128 may be configured to associate identified compliance deficiencies with corresponding tags including F-tags, K-tags, and E-tags corresponding to CMS regulations. The tag association module 128 may use a mapping model to associate deficiency characteristics with applicable regulatory requirements based on semantic similarity. By associating deficiencies with tags that share similar regulatory content, the tag association module 128 enables determination of representative compliance actions that accurately address the identified issues. This targeted association minimizes redundancy and ensures that subsequent compliance processes work with a structured, concise, and semantically enriched subset of the regulatory requirements. In some embodiments, the tag association module 128 associates deficiencies with tags based on one or more semantic concepts/themes such as intent, severity, scope, regulatory domain, affected populations, required interventions, etc.

The scoring module 129 generally calculates risk scores for residents or facilities based on the processed data, user inputs, and normalized compliance metadata. The scoring module 129 may implement dual dynamic risk scoring at both the resident level and the facility level, with scores updating in real time as data is ingested or actions are completed. In some examples, the scoring module 129 may implement regional or other risk scoring subdivisions. The scoring module 129 may correlate diagnostic codes (e.g., ICD-10 codes) with historical citation patterns to weight risk scores. This weighting mechanism may prioritize digital pathways and compliance actions based on quantified risk levels rather than static thresholds. The scoring module 129 may update scores dynamically based on user inputs, audit results, compliance deficiencies, and survey outcome data. The scoring module 129 may learn from uploaded plans of correction and outcome data to improve future recommendations, with prediction accuracy improving over time as more data and outcomes are added. The scoring module 129 may determine survey readiness metrics based on the calculated risk scores and user inputs. In some examples, the scoring module 129 may determine how relevant risk factors are weighted, such as a diagnostic code's correlation with historical citations reaching a threshold, relative to the facility as a whole, etc.

The compliance action module 130 may trigger compliance actions based on the associated tags and detected deficiencies. The compliance action module 130 may be configured to trigger compliance actions including audits, risk score updates, quality improvement recommendations, or mock surveys. The compliance action module 130 may generate audit assessment tools based on identified deficiencies with specific assessment criteria derived from the cited regulatory requirements. The compliance action module 130 may detect triggers within compliance issues and escalate, based on regulatory deadlines, the compliance issues containing triggers. By focusing on the most relevant and actionable compliance requirements, the compliance action module 130 ensures that corrective actions are both targeted and reflective of the core regulatory themes derived from the identified deficiencies.

The compliance action module 130 may include grievance categorization functionality that employs AI keyword scanning to categorize grievances, identify abuse-related triggers, and escalate issues when necessary. The compliance action module 130 may track grievance deadlines, escalate overdue items, and trend grievance categories across time. The compliance action module 130 may also perform rounding analysis where notes entered during staff rounding visits are analyzed with classifiers and embeddings to detect concerns, repeated issues, risk triggers, and potential abuse indicators. These signals may automatically launch grievances, audits, or quality improvement items with prefilled goals, measures, and follow-up cadence.

The compliance action module 130 may further include QAPI automation functionality. The compliance action module 130 may provide QAPI plan creation, meeting minutes, data analysis, and automated Plan-Do-Study-Act (PDSA) cycles. PDSA is a quality improvement methodology where facilities plan an intervention, implement it on a small scale, study the results, and act on what is learned to refine the approach before broader implementation. The compliance action module 130 may review trends from pathways, audits, grievances, rounding notes, mock surveys, and regulatory data and recommend QAPI projects based on emerging patterns. The compliance action module 130 may automatically generate QAPI project recommendations based on detected trends across the compliance data.

In some examples, the compliance action module 130 may include a unified survey command interface. Mock surveys and surveyor mode may include a unified interface or command center for managing the full survey lifecycle. The survey command center functionality may include survey preparation, active survey coordination, findings management, evidence organization, and post-survey workflows. The survey command center may recommend actions such as generating or updating a POC, initiating audits, and assigning follow-up tasks based on survey activity and findings.

The notification interface 145 may include a display, such as a monitor or touchscreen, and a user input device, such as a keyboard, mouse, trackpad, touchscreen, gesture and/or biometric tracking device, or the like. The notification interface 145 may enable a user to interact with a graphical user interface (GUI) for viewing compliance information and providing user selections or inputs. The notification interface 145 may be configured to display one or more interfaces including a pathway sub interface and a dashboard sub interface for presenting survey readiness metrics, plans of correction, risk alerts, and compliance information. The notification interface 145 may enable a user to interact with digital pathways and provide user selections or inputs to the digital pathways.

### Network

The network 150 may include wired and/or wireless communication network(s) such as a cellular network (e.g., 5G®, 4G LTE®, 3G®), a Wi-Fi® network (802.11 standards), a microwave access network (e.g., WiMAX®), and/or any other suitable wide area network (WAN), local area network (LAN), personal area network (PAN), etc. Moreover, the network 150 may be a single communication network, or may include multiple communication networks of one or more types (e.g., one or more wired and/or PANs or LANs, and/or one or more WANs such as the Internet). In some examples, the network 150 includes multiple, entirely distinct networks (e.g., networks for communications between the computing device 110 and the server 120, and a separate, Bluetooth® or wireless LAN (WLAN) network for communications between the user device 140 and another computing device (e.g., smart phone, tablet, 2-in-1 device), and so on). It should be appreciated that, while in the example of FIG. 1 the network 150 is illustrated as a single component, the network 150 may include multiple (e.g., tens, hundreds, or thousands of) networks.

The system 100 may be deployed on a cloud environment using a multi-tenant architecture with API-driven data exchange. The system 100 may enforce encryption at rest and in transit for all data. The system 100 may maintain immutable audit logs of all actions for regulatory chain-of-custody controls.

### Example Operation

In operation, the system 100 may be used to identify compliance deficiencies in healthcare facilities and generate plans of correction for regulatory survey preparation. The data ingestion module 125 may receive, by one or more processors (e.g., the processor 122), structured data (e.g., resident census data containing fields such as admission date, diagnosis codes including ICD-10 codes, medication lists, and care plan elements) via the API. The data ingestion module 125 may also receive unstructured data (e.g., scanned survey statements containing surveyor observations, grievance forms documenting complaints, policy documents specifying facility procedures, and handwritten nursing notes describing care delivery).

The normalization module 127 may prepare the unstructured data through OCR (e.g., extracting text from a scanned document to convert handwritten deficiency observations into machine-readable text) and metadata extraction (e.g., identifying compliance elements such as the cited tag number, scope and severity rating, and specific regulatory requirement violated). The normalization module 127 may convert extracted compliance metadata into standardized formats (e.g., transforming free-text deficiency descriptions into structured JSON objects containing fields for tag identifier, deficiency category, affected residents, and required corrective actions). The normalization module 127 may chunk the extracted text into segments, embed the segments into vector representations (e.g., 1536-dimensional vectors) using an embedding model, and store the embeddings in a vector database for similarity-based retrieval. The system may learn from uploaded POCs and survey outcome data to improve future recommendations. For example, when a facility uploads a successful POC that resulted in citation clearance, the system may store the POC content and associated outcomes in the vector database, enabling retrieval of proven corrective strategies for similar future deficiencies. Prediction accuracy may improve over time as more data and outcomes are added to the system.

The AI processing module 126 may aggregate, using RAG, a prompt for the language model including relevant vector embeddings, a structured template, regulatory logic, and a metadata-specific prompt. The AI processing module 126 may prompt the language model with the structured data and normalized compliance metadata to generate a determination of a compliance deficiency (e.g., analyzing a combination of medication administration records, nursing notes, and grievance data to identify a pattern indicating potential regulatory violations). The AI processing module 126 may generate a complete POC including systemic changes, monitoring plans, responsible roles, due dates, and evidence placeholders.

The tag association module 128 may associate identified compliance deficiencies with corresponding regulatory tags (e.g., mapping a medication error pattern to applicable F-tags) using regulatory logic that correlates deficiency characteristics with the applicable regulatory requirements. The scoring module 129 may calculate risk scores for residents (e.g., assigning a risk score based on fall incidents, medical history, weight loss, etc.) or facilities (e.g., calculating a survey readiness score based on completed pathway assessments, resolved grievances, and audit completion rates). The scoring module 129 may correlate diagnostic codes with historical citation patterns (e.g., determining that residents with certain ICD-10 codes have a higher likelihood of triggering specific citations based on historical survey data).

The compliance action module 130 may trigger compliance actions based on the associated tags and risk scores (e.g., automatically scheduling an audit when the facility risk score exceeds a threshold, generating a quality improvement recommendation when grievance trends indicate recurring complaints, or initiating a mock survey when survey readiness falls below a threshold). The compliance action module 130 may generate audit assessment tools (e.g., creating a customized audit checklist based on identified deficiencies with specific assessment criteria derived from the cited regulatory requirements). The notification interface 145 of the user device 140 may display the pathway sub interface comprising the structured data, normalized compliance metadata, and associated tags (e.g., presenting a digital pathway with pre-populated resident data, required observation fields, and automatic tag associations based on user responses). The notification interface 145 may receive user selections or inputs to digital pathways (e.g., recording observations during a pathway assessment where the user selects options, documents observations, and notes interactions). The dashboard sub interface may display survey readiness metrics, POCs (e.g., presenting the AI-generated POC with collaborative editing capabilities), and risk alerts (e.g., notifying the administrator that a deadline is approaching).

The system 100 may operate as a continuous closed-loop compliance engine where findings from one activity automatically inform subsequent actions. Mock surveys, audits, POCs, QAPI activities, grievances, and events may feed back into risk determination and future system actions. The system 100 may determine what compliance action is required next and initiate that action automatically. For example, mock survey findings may lead to AI POC generation, which may then trigger assigned monitoring, smart audits, QAPI projects, and dashboard updates. The compliance action module 130 may automatically initiate audits and QAPI activities based on findings, events, or risk thresholds. Risk scores, readiness metrics, and recommendations may update in real time as data is ingested or actions are completed, and outcome-based learning may improve future determinations.

In some examples, the system 100 may support AI-driven minimum data set (MDS) validation to detect scoring inconsistencies and omitted items. The MDS is a standardized, federally mandated assessment instrument that nursing homes must complete for each resident to evaluate functional capabilities, health conditions, and care needs. The AI-driven MDS validation may identify when mobility scores are inconsistent with nursing notes. The system 100 may support Patient-Driven Payment Model (PDPM) optimization with automated audits and documentation integrity checks. The PDPM is the Medicare reimbursement system for skilled nursing facilities that bases payment on patient characteristics and care needs rather than volume of services. In some examples, the PDPM optimization may ensure diagnosis coding aligns with clinical documentation for accurate reimbursement. The system 100 may support AI-generated care plan recommendations based on diagnoses, assessments, and survey risk signals. In some examples, the AI-generated care plan recommendations may suggest interventions for residents with elevated risk for that associated tag. The scoring module 129 may learn from uploaded POCs and outcome data to improve future recommendations. For example, when survey outcome data indicates that a particular corrective action successfully resolved a deficiency, the scoring module 129 may weight similar recommendations more heavily in future POC generation. Prediction accuracy may improve over time as more data and outcomes are added, enabling the system to generate increasingly accurate compliance deficiency determinations and risk assessments without requiring retraining of the underlying AI model.

Further, the system 100 may support clinical audit automation across nursing, therapy, wound care, nutrition, and infection control. Nursing clinical audit automation may include medication pass observations. Therapy clinical audit automation may include treatment documentation reviews. Wound care clinical audit automation may include pressure ulcer staging verification. Nutrition clinical audit automation may include weight monitoring and dietary intake tracking. Infection control clinical audit automation may include hand hygiene compliance and isolation precaution adherence. The system 100 may support a quality measure dashboard integrated with the risk engine. In some examples, the quality measure dashboard may correlate quality measure performance with survey citation probability. The system 100 may support a resident-level clinical analytics chatbot for clinical queries. In some cases, the resident-level clinical analytics chatbot may enable staff to ask, “What are the risk factors for Room 201?” and receive AI-generated responses.

Moreover, the system 100 may incorporate expanded data sources (e.g., Epic®, Cerner®, and Meditech® EMRs). In some cases, the system 100 may integrate with hospital EMRs for post-acute transition data. The system 100 may incorporate therapy notes including physical, occupational, and speech therapy documentation. The system 100 may incorporate wound notes including wound care specialist assessments and photography. The system 100 may incorporate social services notes including discharge planning and family communication records. The system 100 may incorporate laboratory results including infection markers and nutritional indicators. The system 100 may incorporate staffing and scheduling systems. In some cases, the system 100 may correlate staffing ratios with compliance outcomes. The expanded data sources may enable comprehensive compliance analysis across all aspects of facility operations.

The architecture of the system 100 may support individual modules to be deployed independently. The individual modules may include a POC-only module for facilities seeking AI-powered POC generation, a pathways-only module for facilities needing digital survey pathway tools, a QAPI-only module for organizations focusing on quality improvement automation, a feedback module for facilities prioritizing resident feedback management, a dashboard module for users needing compliance visibility without operational tools, a smart audit generator module for organizations seeking automated audit tool creation, etc. The modular product packaging may allow organizations to adopt specific functionality based on compliance needs.

### Additional Modules and Functionality

In an example, the present techniques may additionally or alternatively include the following modules and functionality: a mock survey module, an audit module, a grievance module, a CareGuard module, a QAPI module, a POC module, a survey command center module, and a dashboard module.

The mock survey module may digitize CMS survey pathways and inspection processes. The mock survey module may also capture observations, interviews, and record reviews. Further, the mock survey module may identify deficiencies, associate regulatory tags, and generate findings. These findings may prompt recommendations such as initiating or updating a POC, generating an audit, launching monitoring tasks, escalating issues, or initiating QAPI activity.

The audit module may create audit tools dynamically based on risk, findings, operational triggers, or regulatory tags. Audit results may be analyzed and the audit module may generate recommendations to create or update corrective actions, schedule follow-up audits, assign monitoring tasks, and escalate trends to QAPI.

The grievance module may capture and categorize resident, family, and staff grievances. The grievance module may use AI-based pattern detection to identify themes, urgency, potential triggers, and regulatory risk. The grievances may generate recommendations to initiate a corrective action plan, create an audit tool, assign follow-up tasks, escalate issues by deadline, or roll patterns into QAPI projects.

The CareGuard module which may include guardian angels and digital rounding, as further described below, supports structured rounding and resident advocacy workflows. The CareGuard module may capture real-time observations and feedback. As AI continuously analyzes rounding data to identify risks, unmet needs, or trends, the CareGuard module may recommend corrective actions, audits, and follow-up tasks.

The QAPI module may manage QAPI workflows, including PDSA cycles, data tracking, and meeting documentation. QAPI projects may be recommended or initiated based on patterns identified across audits, grievances, mock surveys, rounding data, or risk scoring outputs.

The POC module may provide structured generation, editing, tracking, and validation of corrective action plans. The POC module may include systemic changes, monitoring plans, responsible roles, due dates, and evidence placeholders. The POC module may be a centralized workspace for corrective actions, but it will be understood that corrective actions are not limited to this module, they may be recommended from in a multitude of ways in accordance with the present disclosure.

The survey command center module may manage the end-to-end survey process, including survey preparation, active survey coordination, findings management, evidence organization, and post-survey workflows. The survey command center module may recommend actions such as generating or updating a POC, initiating audits, and assigning follow-up tasks based on survey activity and findings.

The dashboards module may provide real-time visibility across the system (e.g., system 100). Dashboards may aggregate data from any module to display readiness metrics, risk indicators, open findings, recommended actions, deadlines, and status indicators at the facility, regional, and enterprise levels. The dashboard module may function as the connective layer tying any module together.

Across the present techniques, AI may continuously read, analyze, and correlate data generated by any of the modules described herein (e.g., mock survey module, audit module, grievance module, CareGuard module, QAPI module, POC module, survey command center module, dashboard module). Based on this ongoing analysis, the present techniques may produce recommendations and launch workflows including: (1) generating or updating a POC (e.g., a corrective action plan and workflow, regardless of module origin), (2) generating an audit tool and scheduling follow-up audits based on risk or recurring patterns, (3) assigning tasks and monitoring workflows with owners, due dates, and evidence requirements, (4) escalating issues by regulatory deadlines or trigger thresholds, and/or (5) initiating or recommending QAPI projects when trends indicate systemic opportunities for improvement.

Thus, in the present techniques a POC is not limited to a post-survey CMS citation document and is not confined to a single module. Instead, POC may represent the system-wide corrective action framework that can be recommended, generated, updated, and tracked from any part of the platform. The POC module may serve as one interface for creating and managing these corrective actions, but the underlying corrective action construct may be woven through the present techniques as a whole.

### Exemplary Data Processing Flow

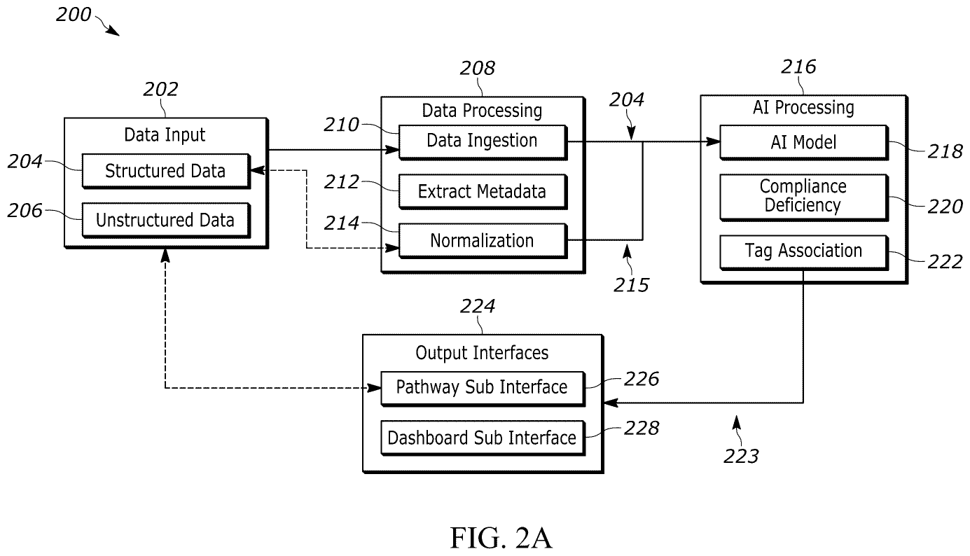

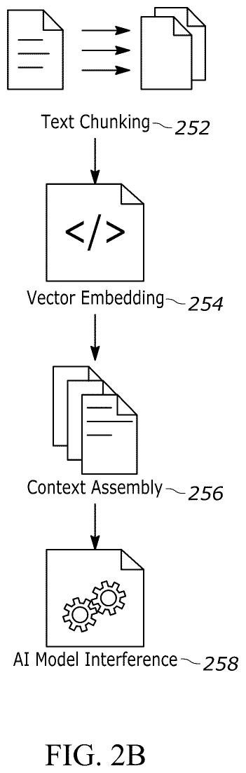

FIG. 2A depicts a flow 200 for processing data of different formats through an artificial intelligence-based system (e.g., the system 100, as described above in reference to FIG. 1). The flow 200 may include a data input 202, a data processing 208, an AI processing 216, and output interfaces 224. The data input 202 may receive data from multiple sources such as EMR systems, CMS databases, uploaded documents, and real-time user entries. The data processing 208 may ingest and normalize received data by converting diverse formats into a unified schema. The AI processing 216 may analyze normalized data and identify compliance deficiencies by detecting patterns indicating regulatory risk. The output interfaces 224 may present processed information to users by displaying actionable insights on dashboards and pathway interfaces. As described herein, a pathway may refer to a structured assessment protocol that guides facility staff through required observations, interviews, and documentation steps corresponding to specific regulatory requirements.

The data input 202 may include structured data 204 and unstructured data 206. The structured data 204 may be received via an API in standardized formats including JSON format such as EMR resident records, XML format such as CMS CASPER exports, CSV format such as quality measure reports, or HL7 format such as clinical messaging data. The unstructured data 206 may be received from users in real time and may include scanned survey statements, photographed policy documents, and voice-to-text notes. The structured data 204 and the unstructured data 206 may include medical data.

The data processing 208 may include a data ingestion 210, an extract metadata 212, and a normalization 214. The data ingestion 210 may integrate with EMR systems (e.g., PointClickCare and MatrixCare) via JSON API to pull daily census updates, medication changes, and care plan modifications. The data ingestion 210 may also ingest CMS data including: CASPER data such as historical survey citations and trends; 802 reports, which are quality measure performance reports that provide facilities with data on specific clinical quality indicators (e.g., pressure ulcers, falls, infections, etc.) for comparison against state and national benchmarks; and Five-Star data such as overall quality ratings via CSV, XML, or API formats. The data ingestion 210 may receive the structured data 204 and the unstructured data 206 and may route the received data for processing by directing EMR data to direct normalization while routing scanned documents through OCR.

The extract metadata 212 may use AWS® Textract or the like as an OCR engine to extract text from PDFs such as policy manuals and consent forms, images such as photographed audit forms and whiteboard schedules, policies such as infection control procedures and fall prevention protocols, grievance forms such as resident complaint documentation, 2567 statements such as survey deficiency reports, and uploaded documents such as staff training records and equipment maintenance logs.

The normalization 214 may produce normalized compliance metadata 215 such as structured JSON objects containing tag identifiers, deficiency categories, and affected resident lists. The normalization 214 may convert extracted compliance metadata into the standardized format by transforming surveyor narrative text into structured deficiency records. The data processing 208 may transmit the normalized compliance metadata 215 to the AI processing 216.

The AI processing 216 may include an AI model 218 and a tag association 222. The AI model 218 may process the structured data 204 and the normalized compliance metadata 215 to generate a compliance deficiency 220 determination. The AI model 218 may generate the compliance deficiency 220 determination by receiving the structured data 204 and the normalized compliance metadata 215 as input to identify compliance deficiencies in the structured data 204 or the normalized compliance metadata 215. The AI model 218 may analyze medication records and nursing notes to identify patterns suggesting violations.

The tag association 222 may associate identified compliance deficiencies with corresponding tags to produce associated tags 223. The associated tags 223 may include F-tags, K-tags, and E-tags. The tags may correspond to one or more CMS regulations. The AI processing 216 may transmit the associated tags 223 to the output interfaces 224.

The output interfaces 224 may include a pathway sub interface 226 and a dashboard sub interface 228. The pathway sub interface 226 may display the structured data 204, the normalized compliance metadata 215, and the associated tags 223 on a graphical user interface (GUI). The pathway sub interface 226 may receive user selections or inputs to a digital pathway. The pathway sub interface 226 may enforce completion of required fields by ensuring all observation criteria are documented before submission. The pathway sub interface 226 may associate user selections or inputs with corresponding tags by linking dining observations to nutrition-related F-tags. The pathway sub interface 226 may present pre-populated infection control pathways with resident-specific data and automatic F-tag linkages.

The dashboard sub interface 228 may display compliance information including medical data such as resident risk scores, facility survey readiness percentages, and open corrective action counts. In some examples, the server 120 may implement the flow 200 by executing the data ingestion module 125, AI processing module 126, normalization module 127, and tag association module 128, as described above in reference to FIG. 1.

### Exemplary AI Processing Pipeline

FIG. 2B depicts an AI processing flow 250 for a data transformation pipeline from raw text input through AI model processing. The AI processing flow 250 may include a text chunking 252, a vector embedding 254, a context assembly 256, and an AI model inference 258. The text chunking 252 may divide input documents into smaller segments for processing. For example, the text chunking 252 may split a 50-page policy manual into sentence-level chunks. The vector embedding 254 may convert text segments into numerical vector representations. For example, the vector embedding 254 may transform regulatory text into mathematical representations capturing semantic meaning. The context assembly 256 may aggregate relevant content with structured templates and regulatory logic. For example, the context assembly 256 may combine retrieved compliance content with POC formatting requirements. The AI model inference 258 may process assembled context and generate compliance outputs. For example, the AI model inference 258 may produce draft POCs, audit checklists, or risk assessments.

The text chunking 252 may divide input text from documents into smaller segments suitable for embedding operations. For example, the text chunking 252 may create chunks (e.g., 512-token chunks) from lengthy survey statements to enable efficient retrieval. The vector embedding 254 may convert the text chunks into vector representations (e.g., 1536-dimensional) using an embedding model for similarity search and retrieval operations. For example, the vector embedding 254 may enable semantic matching between user queries and relevant regulatory content. In some examples, a system (e.g., the system 100, as described above in reference to FIG. 1) may chunk compliance metadata into segments and embed, using an embedding model, the segments into vector representations.

The context assembly 256 may combine relevant vector embeddings with structured templates, regulatory logic, system prompts defining required CMS elements, and metadata-specific prompts using retrieval-augmented generation. For example, the context assembly 256 may combine a number of semantically similar document chunks (e.g., the most semantically similar document chunk), with a CMS-required POC format including five elements, F-tag specific compliance requirements and interpretive guidelines, instructions specifying corrective action, systemic changes, monitoring plan, responsible parties, and completion dates, and facility-specific context (e.g., bed count, survey history, and resident acuity). In some examples, a system (e.g., the system 100, as described above in reference to FIG. 1) may aggregate, using RAG, a prompt for the AI model including relevant vector embeddings, a structured template, regulatory logic, and a metadata-specific prompt. The method for normalizing compliance metadata into a standardized format may include chunking the compliance metadata into segments, embedding the segments into vector representations using an embedding model, and aggregating a prompt for the AI model using RAG.

In some examples, a system (e.g., the system 100, as described above in reference to FIG. 1) may perform post-processing operations including Protected Health Information (PHI) detection using AWS Comprehend Medical. For example, the system may identify and flag patient names, dates of birth, and medical record numbers in AI outputs. The system may calculate confidence scores based on response length using a scale (e.g., 0.0-1.0). For example, the system may assign higher confidence to more detailed, substantive responses. The system may perform text-to-HTML conversion by wrapping outputs in div tags and converting markdown to HTML elements. For example, the system may transform bullet points into proper list elements for display. The system may perform text cleaning that normalizes escape sequences and removes malformed artifacts from AI outputs. For example, the system may clean up errant newline characters or encoding issues.

The system may perform metadata enrichment by adding processing time, document references, chunk counts, and model information to AI outputs. For example, the system may add annotations such as “Generated in 2.3 seconds,” and “Based on 12 source documents,” “Retrieved 8 relevant passages.” The system may perform content validation that detects missing CMS compliance elements and adds default placeholders when needed. For example, the system may alert when a generated POC lacks a monitoring plan component and may insert “[Specify responsible party]” for incomplete fields. The system may use JSON parsing with fallback pattern matching for structured data extraction from AI outputs. For example, the system may extract tag numbers and dates even when JSON formatting is imperfect. The system may collect training data from user feedback, such as recording when users accept, modify, or reject AI-generated content.

### Exemplary Compliance Data Processing Method

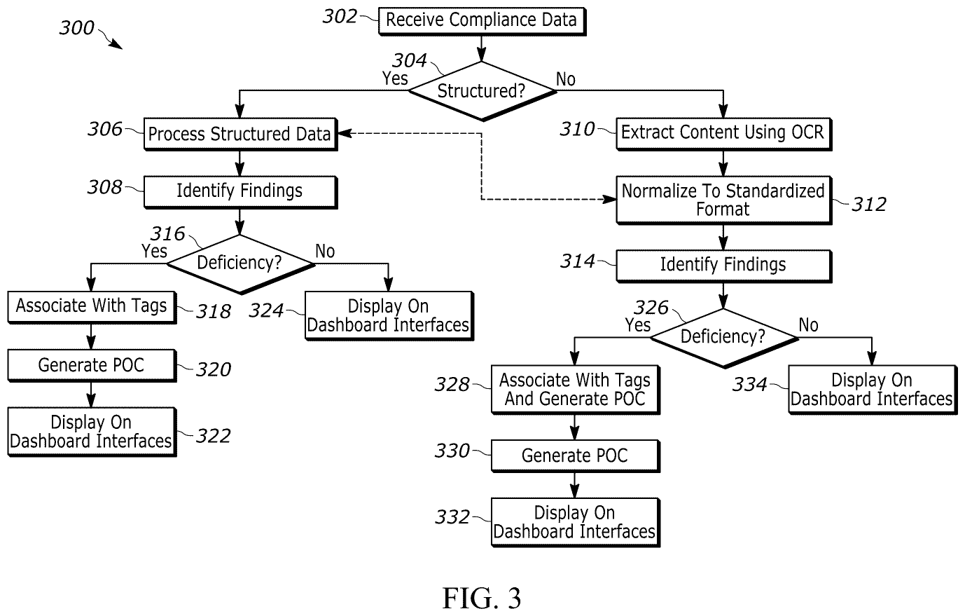

FIG. 3 depicts a method 300 for processing compliance data based on data format determination and deficiency identification. In some examples, a server (e.g., the server 120, as described above in reference to FIG. 1) may perform the method 300, and the method 300 may correspond to the data processing flow (e.g., the flow 200, as described above in reference to FIG. 2A). The method 300 demonstrates a workflow that processes received compliance data through a branching structure that directs the workflow based on whether the data is structured or unstructured, applies OCR extraction and normalization for unstructured data, identifies findings in both paths, and applies secondary branching structures that determine whether deficiencies are associated with tags and POCs are generated before displaying results on dashboard interfaces.

The method 300 may receive compliance data (block 302). The compliance data may include EMR exports, scanned survey documents, uploaded policy files, and real-time pathway observations. The method 300 may determine whether the compliance data is structured (block 304). The determination may include checking if the data contains defined fields and formatting versus free-text content.

If the compliance data is structured (e.g., JSON records from an EMR API, CSV exports from CMS), the method 300 may proceed to process the structured data (block 306). Processing the structured data may include parsing fields, validating data types, and mapping to an internal schema. From block 306, the method 300 may identify findings (block 308). Identifying findings may include detecting patterns indicating potential compliance issues.

If the compliance data is not structured (e.g., scanned 2567 statements, handwritten notes, photographed documents), the method 300 may extract content using OCR (block 310). Extracting content using OCR may include converting image-based text to machine-readable format. From block 310, the method 300 may normalize the extracted content to a standardized format (block 312). Normalizing the extracted content may include transforming free-text observations into structured JSON with tag identifiers, affected residents, and deficiency descriptions. The method 300 may then identify findings from the normalized data (block 314). Identifying findings from the normalized data may include analyzing extracted text for compliance-relevant patterns. A dashed line connects block 312 to block 308, indicating that normalized content from the unstructured data path may also feed into the findings identification process of the structured data path. The connection may enable combining normalized survey observations with structured EMR data for comprehensive analysis.

The method 300 may determine whether a deficiency is present in the structured data path (block 316). The method 300 may determine whether a deficiency is present in the unstructured data path (block 326). The determination may include evaluating whether identified patterns meet thresholds for compliance concern. If a deficiency is present (e.g., medication administration patterns indicating a potential violation), the method 300 may associate the deficiency with tags (block 318 in the structured data path or block 328 in the unstructured data path). Associating the deficiency with tags may include mapping identified issues to specific F-tags, K-tags, or E-tags based on regulatory criteria.

From block 318, the method 300 may generate a POC (block 320). From block 328, the method 300 may generate a POC (block 330). The POC may include systemic changes (e.g., “Implement monthly psychotropic medication review committee”), monitoring plans (e.g., “Pharmacist review of all new psychotropic orders within 48 hours”), responsible roles (e.g., “Director of Nursing and Consultant Pharmacist”), due dates (e.g., “Initial implementation by a target date such as Mar. 15, 2025; ongoing monitoring thereafter”), and evidence placeholders (e.g., “Attach committee meeting minutes, medication review logs, and pharmacist sign-off sheets”).

The method 300 may display results on dashboard interfaces following POC generation in the structured data path (block 322). The method 300 may display results on dashboard interfaces when no deficiency is present in the structured data path (block 324). The method 300 may display results on dashboard interfaces following POC generation in the unstructured data path (block 332). The method 300 may display results on dashboard interfaces when no deficiency is present in the unstructured data path (block 334). Displaying results on dashboard interfaces may include presenting survey readiness scores, open corrective actions, and risk alerts.

In some examples, the method 300 may utilize a computing device (e.g., via a mock survey module) that digitizes the survey process. The present techniques (e.g., using the mock survey module) may replicate CMS survey protocols in digital format. The present techniques may capture findings including deficiency observations with tag associations, observations including dining room assessments and medication pass reviews, and interviews including resident and staff responses. The captured findings, observations, and interviews may update risk scores (e.g., increasing facility risk rating when deficiencies are identified), trigger POC drafts (e.g., generating corrective action plans within seconds of finding entry), launch audits (e.g., scheduling follow-up infection control audits based on identified issues), and populate dashboards (e.g., reflecting mock survey results in real-time survey readiness metrics).

The method 300 may store (e.g., using a digital POC binder) all related files including evidence documents, staff training records, and monitoring logs. The storage of files may allow collaborative editing similar to shared documents, enabling simultaneous review by facility administrators, regional directors, and corporate compliance officers. The storage of files may provide regional and surveyor-level visibility, allowing state surveyors to access organized evidence packages.

In other examples, the method 300 may provide a surveyor mode that allows scoped read-only access so state surveyors may securely review evidence. Surveyor mode may enable accessing organized compliance documentation during survey revisits. Surveyor mode may preserve chain of custody for regulatory compliance purposes, including maintaining audit trails of document access and modifications.

### Exemplary Risk Scoring and Survey Readiness Method

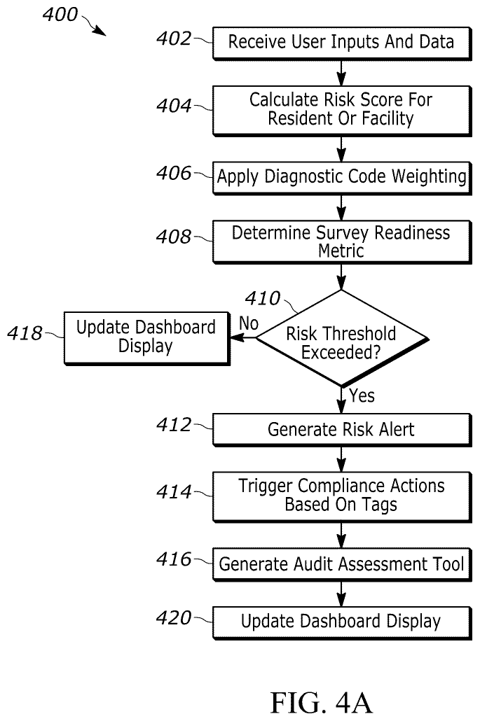

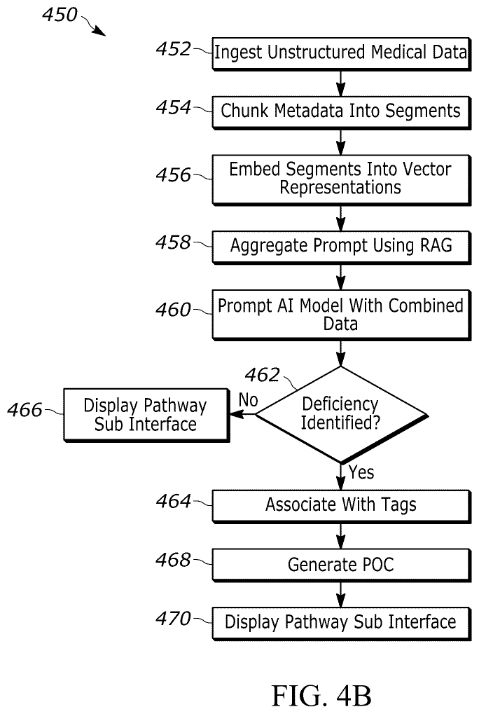

FIG. 4A depicts a method 400 for calculating risk scores and determining survey readiness with conditional compliance action triggering. In some examples, the scoring module 129 and the compliance action module 130, as described above in reference to FIG. 1 may perform the method 400. The method 400 provides a workflow that processes user inputs and data through risk score calculation with diagnostic code weighting, determines survey readiness metrics, and applies a branching structure that directs the workflow based on whether a calculated risk score exceeds a defined threshold.

The method 400 may receive user inputs and data (block 402). The user inputs and data may include user selections or inputs to a digital pathway, structured data received via an API, and normalized compliance metadata extracted from unstructured data. The method 400 may aggregate compliance-related information from multiple sources to establish an initial risk assessment.

The method 400 may calculate a risk score for at least one of a resident or a facility (block 404). The risk score calculation may use clinical and regulatory inputs including pathway findings, audit results, grievances, and compliance deficiencies. In some examples, the scoring module 129, as described above in reference to FIG. 1 may determine the risk score based on the user selections or inputs, the structured data, and the normalized compliance metadata.

The method 400 may apply diagnostic code weighting to the risk score (block 406). The method 400 may incorporate ICD-10 coding into a risk model, assigning priority scores based on diagnoses and condition categories. The present techniques may correlate diagnostic codes with historical citation patterns to weight the risk score. The weighted risk score may drive resident targeting, pathway selection, audit scheduling, and work queues. The scoring module may prioritize, based on the weighted risk score, the digital pathway.

The method 400 may determine a survey readiness metric based on the weighted risk score and the user inputs (block 408). The survey readiness metric may provide a quantitative measure of a facility's preparedness for regulatory surveys. The method 400 may determine the survey readiness metric based on the risk score and the user selections or inputs.

The method 400 may determine if a risk threshold is exceeded (block 410). The method 400 may use customizable risk thresholds (e.g., 1-5, 1-10, or percentage-based scales). Organizations may configure threshold values based on operational requirements and risk tolerance.

If the risk threshold is exceeded at block 410, the method 400 may generate a risk alert (block 412). The method 400 may generate the risk alert based on the risk score and the compliance deficiency. The risk alert may notify facility staff and regional leadership of elevated risk conditions requiring attention.

From block 412, a compliance action module (e.g., the compliance action module 130, as described above in reference to FIG. 1) may trigger, based on associated tags, one or more compliance actions (block 414). The compliance actions may include at least one of an audit, a risk score update, a quality improvement recommendation, or a mock survey. The compliance action module may initiate automated responses to identified compliance deficiencies based on the regulatory tags associated with the deficiencies.

The method 400 may generate an audit assessment tool based on the compliance deficiency or the user selections or inputs (block 416). The audit assessment tool may provide standardized audit structures for evaluating compliance status and tracking corrective actions.

The method 400 may update a dashboard display (block 418 and block 420). The method 400 may update the dashboard display with the risk alert, compliance actions, and audit assessment information when the risk threshold is exceeded (block 420). The method 400 may update the dashboard display without triggering additional compliance actions when the risk threshold is not exceeded (block 418). The one or more interfaces may include a dashboard sub interface comprising the survey readiness metric, a POC, and the risk alert.

The method 400 may prompt the AI model with the structured data and normalized compliance metadata to generate the POC. The POC may include systemic changes, monitoring plans, responsible roles, due dates, and evidence placeholders. The POC may provide a structured corrective action framework aligned with regulatory requirements.

The method 400 may use variable audit frequencies including monthly, weekly, or daily schedules. The method 400 may use QAPI triggers based on user-defined criteria rather than fixed thresholds. The method 400 may support facility-specific or region-specific dashboard configurations, allowing organizations to customize views based on operational needs.

In some examples, the present techniques may update, based on at least one of the user selections or inputs, audit results, the compliance deficiency, or survey outcome data corresponding to a previous compliance deficiency determination, at least one of the risk score, the survey readiness metric, or a vector database. The update functionality enables the system to adapt risk assessments based on new information and historical outcomes.

### Exemplary RAG-Based Unstructured Data Processing Method

FIG. 4B depicts a method 450 for processing unstructured medical data using RAG and deficiency identification. The method 450 transforms unstructured medical data through processing operations including chunking, vector embedding, and RAG to prepare context for AI model inference, with branching logic that determines whether medical compliance deficiencies are identified and how the system responds to such identifications. In some examples, the AI processing module 126 and normalization module 127, as described above in reference to FIG. 1 may perform the method 450. The method 450 may correspond to the AI processing flow (e.g., the AI processing flow 250, as described above in reference to FIG. 2B).

The method 450 may ingest unstructured medical data (block 452). The unstructured medical data may include documents lacking a predefined format, such as scanned images, PDF files, handwritten notes, or free-text entries from various sources within a long-term care facility.

The method 450 may chunk metadata into segments (block 454). The method 450 may divide the ingested unstructured medical data into smaller segments suitable for subsequent processing operations. The method 450 may size the segments to optimize retrieval and model context handling during later stages of the method 450.

The method 450 may embed the segments into vector representations (block 456). The method 450 may use an embedding model to convert the text segments into numerical vector format suitable for similarity search and retrieval operations. The vector representations may enable the system to perform similarity-based comparisons between document segments and user queries.

The method 450 may aggregate a prompt using retrieval-augmented generation (RAG) (block 458). The method 450 may combine relevant vector embeddings with structured templates, regulatory logic, and metadata-specific prompts to create a unified context for AI model processing. The aggregated prompt may include content retrieved from a vector database based on similarity to the input data.

The method 450 may prompt an AI model with combined data (block 460). The AI model may receive the assembled context from the RAG operation and may process the combined data to generate outputs. In some examples, the AI processing module 126, as described above in reference to FIG. 1 may use alternative AI models including rules-based engines, domain-specific transformers, statistical models, or different classifier architectures to perform the processing at block 460.

If a deficiency is identified at block 462 (Yes branch), the method 450 may associate the deficiency with tags (block 464). The tags may include F-tags, K-tags, or E-tags corresponding to one or more regulations. From block 464, the method 450 may generate a plan of correction (POC) (block 468). The POC may include systemic changes, monitoring plans, responsible roles, due dates, and evidence placeholders.

The method 450 may display a pathway sub interface when no deficiency is identified at block 462 (No branch) (block 466). The method 450 may display a pathway sub interface following the generation of the POC at block 468 (block 470). The pathway sub interface presents the processed information and is configured to receive user selections or inputs to a digital pathway.

The system may generate audits through alternative methods including keyword matching, rules engines, template libraries, or hybrid templates plus AI approaches. In some examples, the notification interface 145, as described above in reference to FIG. 1 may use alternative user interface designs including vertical or reorganized dashboard layouts, checklist-based audit interfaces, mobile or tablet applications, or voice or touch-only workflows.

### Exemplary RAG Generation Sequence

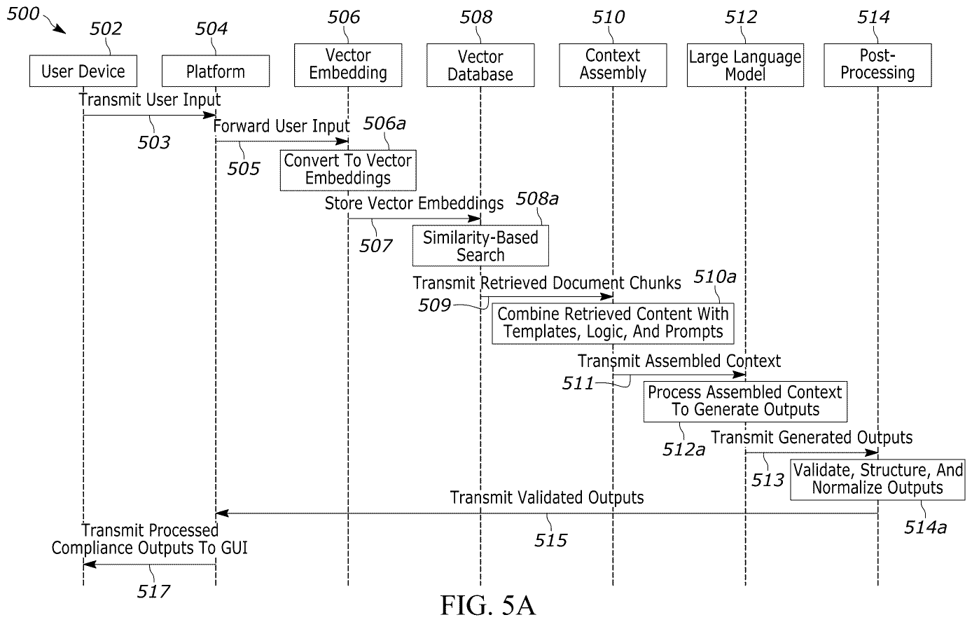

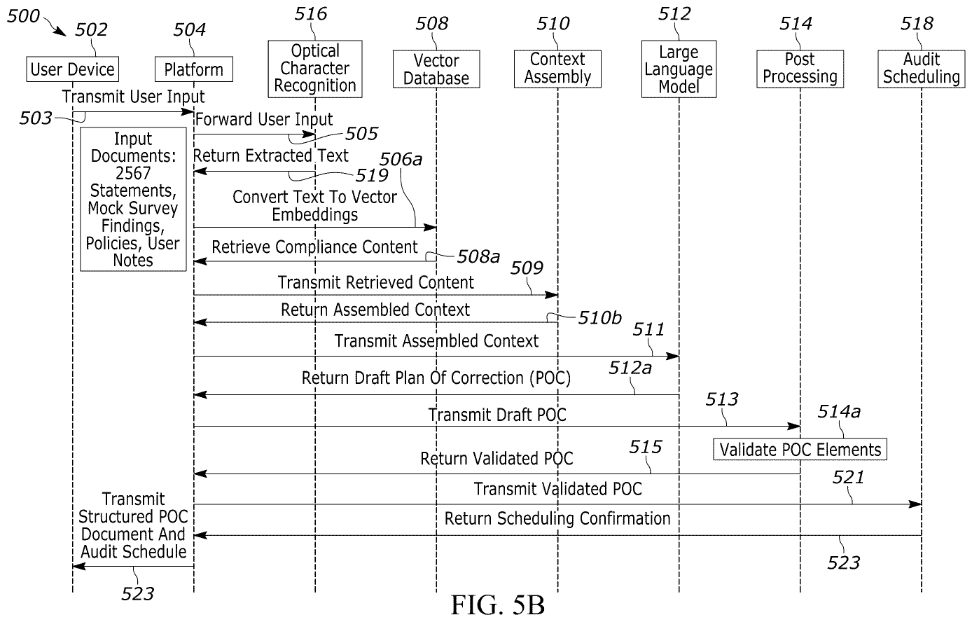

FIG. 5A depicts a process 500 for processing compliance-related data through a RAG pipeline. In some examples, a server (e.g., the server 120, as described above in reference to FIG. 1) may perform operations corresponding to the process 500, and the process 500 may implement aspects of the AI processing flow (e.g., the AI processing flow 250, as described above in reference to FIG. 2B).

The process 500 may include a user device 502, a platform 504, a vector embedding 506, a vector database 508, a context assembly 510, a large language model 512, and post-processing 514. The user device 502 may be associated with facility staff or compliance personnel and may transmit user inputs and receive processed compliance outputs. The platform 504 may coordinate data processing operations between components. The vector embedding 506 may convert text inputs into vector representations. The vector database 508 may store vector embeddings and perform similarity-based searches. The context assembly 510 may combine retrieved content with templates, logic, and prompts. The large language model 512 may process assembled context and generate compliance outputs. The post-processing 514 may validate, structure, and normalize outputs.

The process 500 may include the user device 502 transmitting user input to the platform 504 (operation 503). The platform 504 may forward the user input to the vector embedding 506 (operation 505). The vector embedding 506 may convert the user input to vector embeddings (operation 506a). The vector embedding 506 may store the vector embeddings in the vector database 508 (operation 507). The vector database 508 may perform a similarity-based search to identify relevant document chunks (operation 508a). The vector database 508 may transmit retrieved document chunks to the context assembly 510 (operation 509).

The context assembly 510 may combine retrieved content with templates, logic, and prompts (operation 510a). The context assembly 510 may transmit assembled context to the large language model 512 (operation 511). The large language model 512 may process the assembled context to generate outputs (operation 512a). The large language model 512 may transmit generated outputs to the post-processing 514 (operation 513). The post-processing 514 may validate, structure, and normalize outputs (operation 514 a). The post-processing 514 may transmit validated outputs to the platform 504 (operation 515). The platform 504 may transmit processed compliance outputs to the GUI of the user device 502 (operation 517).

### Exemplary OCR and Audit Scheduling Sequence

FIG. 5B depicts a process 500 for processing compliance-related data through a RAG pipeline with optical character recognition and audit scheduling. The process 500 of FIG. 5B may extend the process 500 of FIG. 5A with additional components. OCR 516 may extract text from input documents including 2567 statements, mock survey findings, policies, and user notes. Audit scheduling 518 may schedule follow-up audits based on validated POC outputs.

The process 500 may include the user device 502 transmitting input documents to the platform 504 (operation 503). The platform 504 may forward input documents to the OCR 516 (operation 505). The OCR 516 may extract text from input documents and return extracted text to the platform 504 (operation 519). The platform 504 may convert extracted text to vector embeddings (operation 506a). The platform 504 may retrieve compliance content from the vector database 508 (operation 508a). The platform 504 may transmit retrieved content to the context assembly 510 (operation 509).

The context assembly 510 may combine retrieved content with templates and prompts and return assembled context (operation 510b). The platform 504 may transmit assembled context to the large language model 512 (operation 511). The large language model 512 may process assembled context and return a draft POC (operation 512a). The post-processing 514 may validate POC elements (operation 514a). The post-processing 514 may return validated POC to the platform 504 (operation 515). The platform 504 may transmit validated POC to the audit scheduling 518 (operation 521). The audit scheduling 518 may return scheduling confirmation (operation 523). The platform 504 may transmit structured POC document and audit schedule to the user device 502 (operation 525).

In some examples, a system (e.g., the system 100, as described above in reference to FIG. 1) may include guardian angel rounding where staff may engage residents regularly and notes may be analyzed with classifiers and embeddings to detect concerns, repeated issues, risk triggers, and potential abuse indicators that may automatically launch grievances, audits, or QAPI items. The system may employ AI keyword scanning to categorize grievances, identify abuse-related triggers, track deadlines, escalate overdue items, and trend categories across time. The system may provide QAPI plan creation, meeting minutes, data analysis, and automated PDSA cycles based on AI-reviewed trends.

### Exemplary Grievance Processing and Escalation Method

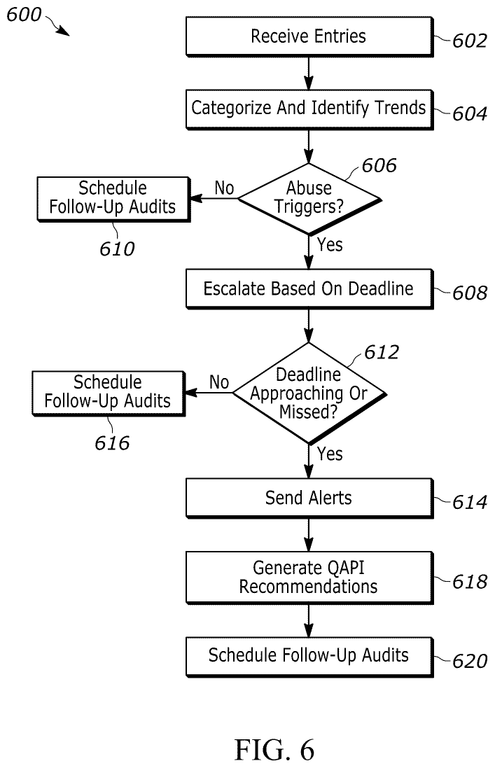

FIG. 6 depicts a method 600 for processing entries and managing compliance actions based on abuse trigger detection and deadline monitoring. In some examples, a compliance action module (e.g., the compliance action module 130, as described above in reference to FIG. 1) may perform the method 600. The method 600 demonstrates a workflow that processes received entries through categorization and trend identification, applies a branching structure based on whether abuse triggers are detected, and applies a secondary branching structure that determines whether deadline-based alerts and quality assurance and performance improvement (QAPI) recommendations are generated before scheduling follow-up audits.

The method 600 may receive entries (block 602). The entries may include grievances such as resident complaints about care quality, family concerns about staff responsiveness, and ombudsman reports. The entries may also include guardian angel rounding notes comprising staff observations during regular resident engagement sessions that document mood changes, physical concerns, or environmental issues.

The method 600 may categorize the entries and identify trends (block 604). The categorization may utilize AI classification to categorize grievances as dietary, nursing care, environmental, or rights related. The method 600 may apply contextual interpretation to detect intent, such as distinguishing between general dissatisfaction and specific care concerns. The method 600 may also detect severity, such as differentiating minor inconveniences from potential harm indicators.

The method 600 may determine whether abuse triggers are detected within compliance issues (block 606). The determination may utilize keyword screening for abuse-related trigger language. The keyword screening may identify terms (e.g., “yelled at,” “ignored,” “left in soiled clothing,” etc.) that may indicate potential abuse or neglect. The system may detect triggers within compliance issues and escalate, based on a regulatory deadline, the compliance issues containing triggers. The method may include detecting triggers within compliance issues and escalating, based on a regulatory deadline, the compliance issues containing triggers.

If abuse triggers are detected at block 606, such as a grievance containing language suggesting mistreatment, the method 600 may escalate based on a regulatory deadline (block 608). The escalation may initiate immediate investigation protocols (e.g., within 24 hours) as may be required by state regulations.

If no abuse triggers are detected at block 606, such as a grievance concerning room temperature preferences, the method 600 may schedule follow-up audits (block 610). The follow-up audits may include adding environmental comfort checks to the next facility audit.

The method 600 may determine whether a deadline is approaching or missed (block 612). The determination may include checking whether a grievance response window (e.g., a 5-day grievance response window), is within a threshold period (e.g., 24 hours) of expiration.

If a deadline is approaching or missed at block 612, such as a grievance response due in 24 hours with no documented resolution, the method 600 may send alerts (block 614). The alerts may notify an administrator (e.g., Director of Nursing) via dashboard notification, email, mobile push notification, etc.

From block 614, the method 600 may generate QAPI recommendations with prefilled objectives and measures (block 618). The prefilled objectives may include “Reduce grievance response time to under 3 days,” and the measures may include “Track percentage of grievances resolved within regulatory timeframes.”

From block 618, the method 600 may schedule follow-up audits (block 620). The follow-up audits may include adding grievance process review to an audit calendar.

If no deadline concern exists at block 612, such as a grievance filed today with response due in a short period (e.g., 5 days), the method 600 may schedule follow-up audits (block 616). The follow-up audits may include incorporating grievance themes into routine audit planning.

The method 600 may utilize a resident targeting mechanism that proposes residents and required pathways using ICD-10 priority, recent events, and grievance signals. The ICD-10 priority may include prioritizing residents with dementia diagnoses for cognitive assessment pathways. The recent events may include flagging residents with recent falls for safety observation pathways. The grievance signals may include directing additional monitoring to residents whose families have filed recent complaints. The resident targeting mechanism may replace blanket audits with risk-based action.

The method 600 may utilize free-text trigger parsing that parses inputs from grievances and guardian angel rounds for trigger terms. The trigger terms may include phrases such as “seems sad,” “not eating,” or “skin looks red” that may indicate emerging concerns. The free-text trigger parsing may auto-classify the trigger terms, such as categorizing “not eating” as nutrition-related and routing to a dietary department. The free-text trigger parsing may launch audits, such as initiating a weight monitoring audit when nutrition concerns are detected. The free-text trigger parsing may launch QAPI items with prefilled goals such as “Improve resident meal consumption rates,” measures such as “Percentage of residents consuming >75% of meals,” and follow-up cadence such as “Weekly checks for identified residents; monthly QAPI review.” The free-text trigger parsing may bring signals into action.

### Exemplary Data Ingestion Method

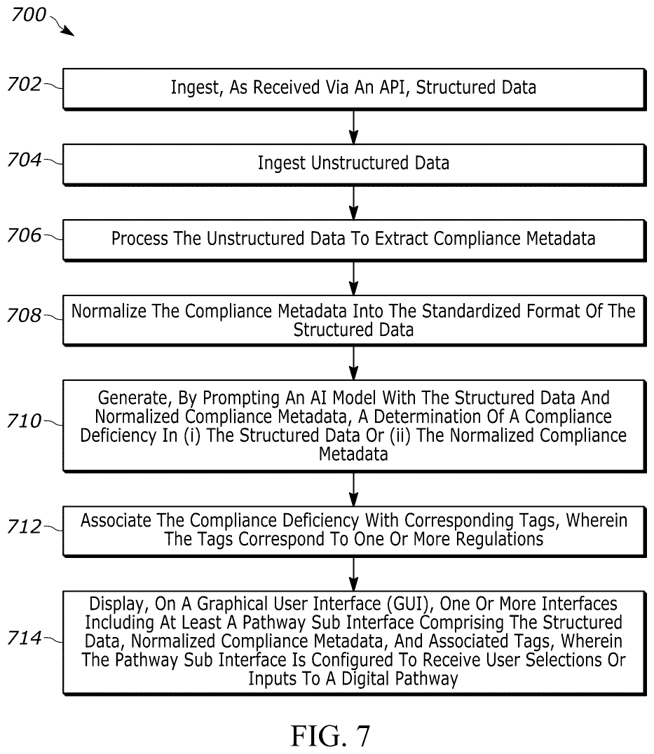

FIG. 7 depicts a method 700 for processing structured and unstructured data to identify compliance deficiencies and display associated information on a graphical user interface. The method 700 demonstrates a data ingestion workflow that transforms data of different formats into compliance outputs. In some examples, a server (e.g., the server 120 as described above in reference to FIG. 1) may perform the method 700. The method 700 may provide an overview of the data processing flow (e.g., the flow 200 as described above in reference to FIG. 2A).

The method 700 may ingest structured data as received via an API in standardized formats (block 702). In some examples, the structured data may include JSON-formatted resident records from an electronic medical record system such as PointClickCare, XML-formatted survey data from CMS CASPER, or CSV-formatted quality measure reports from health departments.

The method 700 may ingest unstructured data (block 704). The unstructured data may include scanned 2567 survey statements uploaded by administrators, photographed nursing notes captured via tablet, voice-recorded observations transcribed to text, or emailed policy documents forwarded to the system.

The method 700 may process the unstructured data to extract compliance metadata (block 706). In some examples, the processing may include applying OCR to convert scanned text to machine-readable format, then identifying elements such as F-tag numbers, deficiency descriptions, and affected resident counts.

The method 700 may normalize the compliance metadata into the standardized format of the structured data (block 708). The normalizing may include transforming extracted text into JSON objects with consistent field names, data types, and value formats matching the structured data schema.

The method 700 may generate, by prompting an AI model with the structured data and normalized compliance metadata, a determination of a compliance deficiency in (i) the structured data or (ii) the normalized compliance metadata (block 710). In some examples, the generating may include submitting combined EMR data and survey history to a large language model with instructions to identify patterns indicating regulatory risk.

The method 700 may associate the compliance deficiency with corresponding tags, wherein the tags correspond to one or more regulations (block 712). The associating may include mapping identified medication administration patterns to a tag for unnecessary drugs, another tag for psychotropic medications, or yet another tag for medication regimen review based on the deficiency characteristics.

The method 700 may display, on a graphical user interface (GUI), one or more interfaces including at least a pathway sub interface comprising the structured data, normalized compliance metadata, and associated tags, wherein the pathway sub interface is configured to receive user selections or inputs to a digital pathway (block 714). The structured data may include resident demographics and clinical information. The normalized compliance metadata may include extracted survey findings and deficiency descriptions. The associated tags may include linked F-tags with regulatory requirements and interpretive guidance. In some examples, the pathway sub interface may enable facility staff to document observations, record interview responses, and upload supporting evidence as the facility staff complete compliance assessments.

### Exemplary Multi-Facility Compliance Architecture

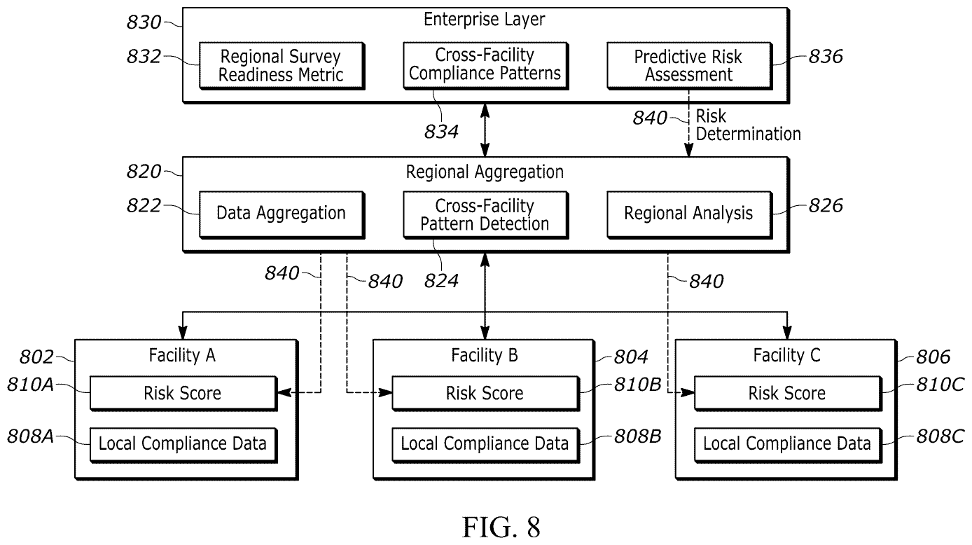

FIG. 8 is a block diagram depicting a hierarchical architecture for managing regulatory risk across multiple long-term care facilities. In some examples, multiple instances of a system (e.g., the system 100 as described above in reference to FIG. 1) deployed across a multi-facility organization may implement the architecture of FIG. 8. As an illustrative example, consider a multi-facility skilled nursing chain operating across multiple states.

The architecture of FIG. 8 may include a facility layer, regional aggregation 820 layer, and enterprise layer 830. The facility layer may include a facility A 802, facility B 804, and facility C 806. The facility layer may be configured to capture local findings such as mock survey observations and pathway assessments, tasks such as assigned corrective actions and pending audits, grievances such as resident complaints and family concerns, audits such as infection control reviews and medication pass observations, and POCs such as active plans of correction with completion status.

The regional aggregation 820 layer may be associated with multi-facility oversight. In some examples, the regional aggregation 820 layer may be associated with a user (e.g., regional director) responsible for facilities in a geographic region (e.g., the Midwest). The regional aggregation 820 layer may be configured to aggregate data across facilities and provide comparative analytics. The comparative analytics may include comparing survey readiness scores, citation rates, and deficiency trends across the region.

The enterprise layer 830 may be associated with corporate-level oversight. In some examples, the enterprise layer 830 may be associated with an organization's leadership (e.g., Chief Compliance Officer, executive leadership team, etc.). The enterprise layer 830 may be configured to provide enterprise-wide compliance intelligence. The enterprise-wide compliance intelligence may include identifying systemic risks affecting the entire organization and benchmarking performance against industry standards.

Each facility in the facility layer may include local compliance data and a risk score. The facility A 802 includes local compliance data 808A and a risk score 810A. The facility B 804 includes local compliance data 808B and a risk score 810B. The facility C 806 includes local compliance data 808C and a risk score 810C. The local compliance data 808A, 808B, 808C may be configured to store compliance information such as survey history, audit results, grievance records, and POC documentation. The risk score 810A, 810B, 810C may be configured to reflect compliance status. In some examples, the risk score 810A, 810B, 810C may comprise a score (e.g., 0-100) indicating survey readiness with higher scores representing lower regulatory risk.

The regional aggregation 820 may include a data aggregation 822, a cross-facility pattern detection 824, and a regional analysis 826. The data aggregation 822 may be configured to consolidate compliance information from all facilities. In some examples, the data aggregation 822 may combine survey readiness data from 15 facilities into regional dashboards. The cross-facility pattern detection 824 may be configured to identify patterns and trends visible when comparing data across multiple facilities such as common F-tag citations across buildings. In some examples, the cross-facility pattern detection 824 may detect that a subset of facilities have elevated F-tag risk. The cross-facility pattern detection 824 may enable data from one facility to protect other facilities by identifying emerging compliance risks before they spread. In some examples, the cross-facility pattern detection 824 may alert the facility B 804 to implement preventive measures after similar deficiency patterns were identified at the facility A 802. Patterns identified at one facility may trigger preventive actions at other facilities before similar issues arise.

For example, if the facility A 802 receives an infection control citation, the cross-facility pattern detection 824 may automatically flag infection control pathways for priority completion at the facility B 804 and the facility C 806. The cross-facility pattern detection 824 may use embedding models to compare residents across facilities and detect cross-facility patterns. For example, the embedding models may generate vector representations of resident characteristics, diagnoses, and care patterns, enabling similarity-based comparison across the multi-facility dataset to identify residents at elevated risk based on patterns observed at other facilities. The regional analysis 826 may be configured to perform analytical operations on aggregated data to generate regional-level insights. In some examples, the regional analysis 826 may identify that facilities with higher staff turnover correlate with increased infection control citations.

The enterprise layer 830 may include a regional survey readiness metric 832, cross-facility compliance patterns 834, and a predictive risk assessment 836. The regional survey readiness metric 832 may be configured to provide an enterprise-level view of survey preparedness across all facilities. In some examples, the regional survey readiness metric 832 may show that a first region averages a first readiness score while a second region averages a second readiness score. The cross-facility compliance patterns 834 may be configured to present patterns identified across the multi-facility dataset. In some examples, the cross-facility compliance patterns 834 may reveal that particular citations have increased or decreased over time periods. The predictive risk assessment 836 may be configured to generate predictions regarding regulatory risk. In some examples, the predictive risk assessment 836 may forecast which facilities have elevated probability of Immediate Jeopardy (IJ) citations in the next 90 days. Accuracy of the predictive risk assessment 836 may improve as more facilities and outcomes are added. In some examples, prediction accuracy may increase as the system 100 learns from survey outcomes. The predictive risk assessment 836 may use data from one facility to predict or mitigate risk in other facilities. For example, when the facility A 802 experiences a citation pattern, the predictive risk assessment 836 may increase risk scores for facilities with similar resident populations, staffing patterns, or operational characteristics.

The facility layer may transmit local compliance data to the regional aggregation 820. In some examples, the facility layer may automatically synchronize daily updates including new audit findings, grievance entries, and POC progress. The data aggregation 822 may consolidate compliance information from all facilities. In some examples, the data aggregation 822 may merge data streams from 15 facilities into unified regional views. The cross-facility pattern detection 824 may analyze aggregated data to identify cross-facility patterns. In some examples, the cross-facility pattern detection 824 may detect emerging citation trends before the emerging citation trends become widespread. The regional analysis 826 may generate insights from aggregated and pattern data. In some examples, the regional analysis 826 may produce weekly regional compliance briefings highlighting areas requiring attention. The enterprise layer 830 may receive analyzed data and generate enterprise-level metrics and predictions. In some examples, the enterprise layer 830 may calculate organization-wide survey readiness and project quarterly citation exposure.

A risk determination 840 may provide a feedback mechanism where enterprise-level risk assessments flow back to update individual facility risk scores 810A, 810B, 810C. In some examples, the risk determination 840 may increase the risk score 810C of the facility C 806 when enterprise analysis identifies the facility C 806 as an outlier on infection control metrics. The risk determination 840 demonstrates that enterprise-level risk assessments may inform and update the individual facility risk scores.

### Exemplary Aspects

The following non-limiting aspects provide example embodiments of the disclosure.

Aspect 1. An artificial intelligence (AI) based system configured to ingest data of different formats, comprising: one or more processors; an AI model programmatically accessible by the one or more processors; an application programming interface (API) configured to receive structured data comprising a standardized format; and one or more memories communicatively coupled to the one or more processors storing computing instructions that, when executed by the one or more processors, cause the one or more processors to: ingest, as received via the API, the structured data; ingest unstructured data; process the unstructured data to extract compliance metadata; normalize the compliance metadata into the standardized format of the structured data; generate, by prompting the AI model with the structured data and normalized compliance metadata, a determination of a compliance deficiency in (i) the structured data or (ii) the normalized compliance metadata; associate the compliance deficiency with corresponding tags, wherein the tags correspond to one or more regulations; and display, on a graphical user interface (GUI), one or more interfaces including at least a pathway sub interface comprising the structured data, normalized compliance metadata, and associated tags, wherein the pathway sub interface is configured to receive user selections or inputs to a digital pathway.

Aspect 2. The artificial intelligence (AI) based system of aspect 1, further comprising computing instructions that cause the one or more processors to: determine based on (i) the user selections or inputs, (ii) structured data, and (iii) normalized compliance metadata, a risk score for at least one of a resident or a facility; determine, based on the risk score and the user selections or inputs, a survey readiness metric; generate, by prompting the AI model with the structured data and normalized compliance metadata, a plan of correction (POC); and generate, based on the risk score and the compliance deficiency, a risk alert, wherein the one or more interfaces includes a dashboard sub interface comprising the survey readiness metric, the POC, and the risk alert.

Aspect 3. The artificial intelligence (AI) based system of any one of aspects 1-2, further comprising computing instructions that cause the one or more processors to: correlate diagnostic codes with historical citation patterns; weight, based on the correlated diagnostic codes and historical citation patterns, the risk score; prioritize, based on the weighted risk score, the digital pathway; and update, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database.

Aspect 4. The artificial intelligence (AI) based system of any one of aspects 1-3, further comprising computing instructions that cause the one or more processors to: trigger, based on the associated tags, one or more compliance actions including at least one of (i) an audit, (ii) a risk score update, (iii) a quality improvement recommendation, or (iv) a mock survey; and generate an audit assessment tool based on the compliance deficiency or the user selections or inputs.

Aspect 5. The artificial intelligence (AI) based system of any one of aspects 1-4, wherein normalizing the compliance metadata into the standardized format further comprises: chunking the compliance metadata into segments; embedding, using an embedding model, the segments into vector representations; and aggregating, using retrieval-augmented generation (RAG), a prompt for the AI model including (i) relevant vector embeddings, (ii) a structured template, (iii) regulatory logic, and (iv) a metadata-specific prompt.

Aspect 6. The artificial intelligence (AI) based system of any one of aspects 1-5, wherein the pathway sub interface is configured to (i) enforce completion of a required field before the user selections or inputs and (ii) associate the user selections or inputs with the corresponding tags, and wherein the tags include at least one of an F-tag, a K-tag, or an E-tag.

Aspect 7. The artificial intelligence (AI) based system of any one of aspects 1-6, wherein the POC includes systemic changes, monitoring plans, responsible roles, due dates, and evidence placeholders.

Aspect 8. The artificial intelligence (AI) based system of any one of aspects 1-7, further comprising computing instructions that cause the one or more processors to: detect triggers within compliance issues; and escalate, based on a regulatory deadline, the compliance issues containing triggers.

Aspect 9. The artificial intelligence (AI) based system of any one of aspects 1-8, wherein the standardized format comprises at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

Aspect 10. A method for artificial intelligence (AI) based data ingestion of different formats, comprising: ingesting, by one or more processors via an application programming interface (API), structured data comprising a standardized format; ingesting, by the one or more processors, unstructured data; processing, by the one or more processors, the unstructured data to extract compliance metadata; normalizing, by the one or more processors, the compliance metadata into the standardized format of the structured data; generating, by prompting an AI model with the structured data and normalized compliance metadata, a determination of a compliance deficiency in (i) the structured data or (ii) the normalized compliance metadata; associating, by the one or more processors, the compliance deficiency with corresponding tags, wherein the tags correspond to one or more regulations; and displaying, on a graphical user interface (GUI), one or more interfaces including at least a pathway sub interface comprising the structured data, normalized compliance metadata, and associated tags, wherein the pathway sub interface is configured to receive user selections or inputs to a digital pathway.

Aspect 11. The method of aspect 10, further comprising: determining, based on (i) the user selections or inputs, (ii) structured data, and (iii) normalized compliance metadata, a risk score for at least one of a resident or a facility; determining, based on the risk score and the user selections or inputs, a survey readiness metric; generating, by prompting the AI model with the structured data and normalized compliance metadata, a plan of correction (POC); and generating, based on the risk score and the compliance deficiency, a risk alert, wherein the one or more interfaces includes a dashboard sub interface comprising the survey readiness metric, the POC, and the risk alert.

Aspect 12. The method of any one of aspects 10-11, further comprising: correlating diagnostic codes with historical citation patterns; weighting, based on the correlated diagnostic codes and historical citation patterns, the risk score; prioritizing, based on the weighted risk score, the digital pathway; and updating, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database.

Aspect 13. The method of any one of aspects 10-12, further comprising: triggering, based on the associated tags, one or more compliance actions including at least one of (i) an audit, (ii) a risk score update, (iii) a quality improvement recommendation, or (iv) a mock survey; and generating an audit assessment tool based on the compliance deficiency or the user selections or inputs.

Aspect 14. The method of any one of aspects 10-13, wherein normalizing the compliance metadata into the standardized format further comprises: chunking the compliance metadata into segments; embedding, using an embedding model, the segments into vector representations; and aggregating, using retrieval-augmented generation (RAG), a prompt for the AI model including (i) relevant vector embeddings, (ii) a structured template, (iii) regulatory logic, and (iv) a metadata-specific prompt.

Aspect 15. The method of any one of aspects 10-14, wherein the pathway sub interface is configured to (i) enforce completion of a required field before the user selections or inputs and (ii) associate the user selections or inputs with the corresponding tags, and wherein the tags include at least one of an F-tag, a K-tag, or an E-tag.

Aspect 16. The method of any one of aspects 10-15, wherein the POC includes systemic changes, monitoring plans, responsible roles, due dates, and evidence placeholders.

Aspect 17. The method of any one of aspects 10-16, further comprising: detecting triggers within compliance issues; and escalating, based on a regulatory deadline, the compliance issues containing triggers.

Aspect 18. The method of any one of aspects 10-17, wherein the standardized format comprises at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

Aspect 19. A tangible, non-transitory computer-readable medium storing instructions for artificial intelligence (AI) based data ingestion of different formats, that when executed by one or more processors cause the one or more processors to: ingest, via an application programming interface (API), structured data comprising a standardized format; ingest unstructured data; process the unstructured data to extract compliance metadata; normalize the compliance metadata into the standardized format of the structured data; generate, by prompting an AI model with the structured data and normalized compliance metadata, a determination of a compliance deficiency in (i) the structured data or (ii) the normalized compliance metadata; associate the compliance deficiency with corresponding tags, wherein the tags correspond to one or more regulations; and display, on a graphical user interface (GUI), one or more interfaces including at least a pathway sub interface comprising the structured data, normalized compliance metadata, and associated tags, wherein the pathway sub interface is configured to receive user selections or inputs to a digital pathway.

Aspect 20. The tangible, non-transitory computer-readable medium of aspect 19, wherein the instructions further cause the one or more processors to: determine, based on (i) the user selections or inputs, (ii) structured data, and (iii) normalized compliance metadata, a risk score for at least one of a resident or a facility; determine, based on the risk score and the user selections or inputs, a survey readiness metric; generate, by prompting the AI model with the structured data and normalized compliance metadata, a plan of correction (POC); and generate, based on the risk score and the compliance deficiency, a risk alert, wherein the one or more interfaces includes a dashboard sub interface comprising the survey readiness metric, the POC, and the risk alert.

### Additional Considerations

Machine readable storage including machine-readable instructions, when executed, to implement a method or realize an apparatus in any of the examples of the present application.

Various techniques, or certain aspects or portions thereof, may take the form of program code (i.e., instructions) embodied in tangible media, such as floppy diskettes, CD-ROMs, hard drives, a non-transitory computer readable storage medium, or any other machine-readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. The eNB (or other base station) and UE (or other mobile station) may also include a transceiver component, a counter component, a processing component, and/or a clock component or timer component. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or an interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to “an example” means that a particular feature, structure, or characteristic described in connection with the example is included in at least one embodiment of the present invention. Thus, appearances of the phrase “in an example” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

Those having skill in the art will appreciate that many changes may be made to the details of the above-described embodiments without departing from the underlying principles of the invention. The scope of the present invention should, therefore, be determined only by the following claims.

What is claimed is:

1. An artificial intelligence (AI) based system configured to ingest data of different formats, comprising:
one or more processors;
an AI model programmatically accessible by the one or more processors;
an application programming interface (API) configured to receive structured data comprising a standardized format; and
one or more memories communicatively coupled to the one or more processors storing computing instructions that, when executed by the one or more processors, cause the one or more processors to:
ingest, as received via the API, the structured data;
ingest unstructured data;
process the unstructured data to extract compliance metadata;
normalize the compliance metadata into the standardized format of the structured data;
generate, by prompting the AI model with the structured data and normalized compliance metadata, a determination of a compliance deficiency in (i) the structured data or (ii) the normalized compliance metadata;
associate the compliance deficiency with corresponding tags, wherein the tags correspond to one or more regulations; and
display, on a graphical user interface (GUI), one or more interfaces including at least a pathway sub interface comprising the structured data, normalized compliance metadata, and associated tags,
wherein the pathway sub interface is configured to receive user selections or inputs to a digital pathway.

2. The artificial intelligence (AI) based system of claim 1, further comprising computing instructions that cause the one or more processors to:
determine based on (i) the user selections or inputs, (ii) structured data, and (iii) normalized compliance metadata, a risk score for at least one of a resident or a facility;
determine, based on the risk score and the user selections or inputs, a survey readiness metric;
generate, by prompting the AI model with the structured data and normalized compliance metadata, a plan of correction (POC); and
generate, based on the risk score and the compliance deficiency, a risk alert, wherein the one or more interfaces includes a dashboard sub interface comprising the survey readiness metric, the POC, and the risk alert.

3. The artificial intelligence (AI) based system of claim 2, further comprising computing instructions that cause the one or more processors to:
correlate diagnostic codes with historical citation patterns;
weight, based on the correlated diagnostic codes and historical citation patterns, the risk score;
prioritize, based on the weighted risk score, the digital pathway; and
update, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database.

4. The artificial intelligence (AI) based system of claim 2, further comprising computing instructions that cause the one or more processors to:
trigger, based on the associated tags, one or more compliance actions including at least one of (i) an audit, (ii) a risk score update, (iii) a quality improvement recommendation, or (iv) a mock survey; and
generate an audit assessment tool based on the compliance deficiency or the user selections or inputs.

5. The artificial intelligence (AI) based system of claim 1, wherein normalizing the compliance metadata into the standardized format further comprises:
chunking the compliance metadata into segments;
embedding, using an embedding model, the segments into vector representations; and
aggregating, using retrieval-augmented generation (RAG), a prompt for the AI model including (i) relevant vector embeddings, (ii) a structured template, (iii) regulatory logic, and (iv) a metadata-specific prompt.

6. The artificial intelligence (AI) based system of claim 1, wherein the pathway sub interface is configured to (i) enforce completion of a required field before the user selections or inputs and (ii) associate the user selections or inputs with the corresponding tags, and wherein the tags include at least one of an F-tag, a K-tag, or an E-tag.

7. The artificial intelligence (AI) based system of claim 2, wherein the POC includes systemic changes, monitoring plans, responsible roles, due dates, and evidence placeholders.

8. The artificial intelligence (AI) based system of claim 1, further comprising computing instructions that cause the one or more processors to:
detect triggers within compliance issues; and
escalate, based on a regulatory deadline, the compliance issues containing triggers.

9. The artificial intelligence (AI) based system of claim 1, wherein the standardized format comprises at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

10. A method for artificial intelligence (AI) based data ingestion of different formats, comprising:
ingesting, by one or more processors via an application programming interface (API), structured data comprising a standardized format;
ingesting, by the one or more processors, unstructured data;
processing, by the one or more processors, the unstructured data to extract compliance metadata;
normalizing, by the one or more processors, the compliance metadata into the standardized format of the structured data;
generating, by prompting an AI model with the structured data and normalized compliance metadata, a determination of a compliance deficiency in (i) the structured data or (ii) the normalized compliance metadata;
associating, by the one or more processors, the compliance deficiency with corresponding tags, wherein the tags correspond to one or more regulations; and
displaying, on a graphical user interface (GUI), one or more interfaces including at least a pathway sub interface comprising the structured data, normalized compliance metadata, and associated tags,
wherein the pathway sub interface is configured to receive user selections or inputs to a digital pathway.

11. The method of claim 10, further comprising:
determining, based on (i) the user selections or inputs, (ii) structured data, and (iii) normalized compliance metadata, a risk score for at least one of a resident or a facility;
determining, based on the risk score and the user selections or inputs, a survey readiness metric;
generating, by prompting the AI model with the structured data and normalized compliance metadata, a plan of correction (POC); and
generating, based on the risk score and the compliance deficiency, a risk alert, wherein the one or more interfaces includes a dashboard sub interface comprising the survey readiness metric, the POC, and the risk alert.

12. The method of claim 11, further comprising:
correlating diagnostic codes with historical citation patterns;
weighting, based on the correlated diagnostic codes and historical citation patterns, the risk score;
prioritizing, based on the weighted risk score, the digital pathway; and
updating, based on at least one of (i) the user selections or inputs, (ii) audit results, (iii) the compliance deficiency, or (iv) survey outcome data corresponding to a previous compliance deficiency determination, at least one of the risk score, the survey readiness metric, or a vector database.

13. The method of claim 11, further comprising:
triggering, based on the associated tags, one or more compliance actions including at least one of (i) an audit, (ii) a risk score update, (iii) a quality improvement recommendation, or (iv) a mock survey; and
generating an audit assessment tool based on the compliance deficiency or the user selections or inputs.

14. The method of claim 10, wherein normalizing the compliance metadata into the standardized format further comprises:
chunking the compliance metadata into segments;
embedding, using an embedding model, the segments into vector representations; and
aggregating, using retrieval-augmented generation (RAG), a prompt for the AI model including (i) relevant vector embeddings, (ii) a structured template, (iii) regulatory logic, and (iv) a metadata-specific prompt.

15. The method of claim 10, wherein the pathway sub interface is configured to (i) enforce completion of a required field before the user selections or inputs and (ii) associate the user selections or inputs with the corresponding tags, and wherein the tags include at least one of an F-tag, a K-tag, or an E-tag.

16. The method of claim 11, wherein the POC includes systemic changes, monitoring plans, responsible roles, due dates, and evidence placeholders.

17. The method of claim 10, further comprising:
detecting triggers within compliance issues; and
escalating, based on a regulatory deadline, the compliance issues containing triggers.

18. The method of claim 10, wherein the standardized format comprises at least one of JavaScript Object Notation (JSON) format, eXtensible Markup Language (XML) format, Comma Separated Value (CSV) format, or Health Level Seven (HL7) format, wherein (i) the structured data or (ii) unstructured data includes medical data, and wherein the unstructured data is received from a user in real time.

19. A tangible, non-transitory computer-readable medium storing instructions for artificial intelligence (AI) based data ingestion of different formats, that when executed by one or more processors cause the one or more processors to:
ingest, via an application programming interface (API), structured data comprising a standardized format;
ingest unstructured data;
process the unstructured data to extract compliance metadata;
normalize the compliance metadata into the standardized format of the structured data;
generate, by prompting an AI model with the structured data and normalized compliance metadata, a determination of a compliance deficiency in (i) the structured data or (ii) the normalized compliance metadata;
associate the compliance deficiency with corresponding tags, wherein the tags correspond to one or more regulations; and
display, on a graphical user interface (GUI), one or more interfaces including at least a pathway sub interface comprising the structured data, normalized compliance metadata, and associated tags,
wherein the pathway sub interface is configured to receive user selections or inputs to a digital pathway.

20. The tangible, non-transitory computer-readable medium of claim 19, wherein the instructions further cause the one or more processors to:
determine, based on (i) the user selections or inputs, (ii) structured data, and (iii) normalized compliance metadata, a risk score for at least one of a resident or a facility;
determine, based on the risk score and the user selections or inputs, a survey readiness metric;
generate, by prompting the AI model with the structured data and normalized compliance metadata, a plan of correction (POC); and
generate, based on the risk score and the compliance deficiency, a risk alert, wherein the one or more interfaces includes a dashboard sub interface comprising the survey readiness metric, the POC, and the risk alert.
